(19) United States
(12) Patent Application Publication
(10) Pub. No.: US 2020/0030629 A1
(43) Pub. Date: Jan. 30, 2020

(54) BOVINE HEADPIECE AND METHOD

(71) Applicant: EQUILUME LTD., Kildare (IE)

(72) Inventor: Barbara MURPHY, Sallins (IE)

(21) Appl. No.: 16/487,970

(22) PCT Filed: Feb. 22, 2018
(86) PCT No.: PCT/EP2018/054446

§ 371 Date: Aug. 22, 2019

(30) Foreign Application Priority Data
Feb. 22, 2017 (GB) 1702821.8
Jun. 26, 2017 (GB) 1710144.5

(51) Int. Cl. A61N 5/06; A61D 99/00

(52) U.S. Cl. CPC A61N 5/0622; A61D 99/00; A61N 2005/0667; A61N 2005/0663; A61N 2005/0651; A61N 2005/0648

(57) ABSTRACT

There is disclosed a light-based method of causing a substantial suppression of melatonin production, the method comprising the step of shining blue light from an artificial blue light source into just one eye of a bovine, causing a substantial suppression of melatonin production in the bovine, sufficient to induce physiological change. There is further disclosed a device for causing a substantial suppression of melatonin production, the device comprising a headpiece fittable to a bovine's head, and including an artificial blue light source, the headpiece operable to shine blue light from the artificial blue light source into an eye of the bovine, causing a substantial suppression of melatonin production in the bovine, sufficient to induce physiological change.

### BACKGROUND OF THE INVENTION

### 1. Field of the Invention

The field of the invention relates to an apparatus and to a method for suppressing melatonin synthesis in a bovine.

### 2. Technical Background

Photoperiod manipulation is widely used within the Agri-Food sector to increase production and efficiency, including for dairy cows (Dahl and Petitclerc, 2003). The first observation that artificially extending daylight hours could influence milk production in lactating dairy cows was made in 1978 (Peters et al., 1978) and since then at least nine other studies from various laboratories across two continents have supported these initial findings (Peters et al., 1981; Marcek and Swanson, 1983; Stanisiewski et al., 1985; Bilodeau et al., 1989; Evans and Hacker, 1989; Phillips and Schofield, 1989; Dahl et al., 1997; Miller et al., 1999; Porter and Luhman, 2002: FIG. 1). All studies reiterated that providing lactating cows with 16-18 hours (h) of light per day (d) can increase milk yield by approximately 2 kg per day.

At the core of the physiological mechanism for increased milk production in response to extended daylength is regulation by light of the pineal hormone melatonin (MT). The approximate 24 hours (circadian) rhythm of MT secretion from the pineal gland is one of the most stable outputs from the circadian system (Benloucif et al., 2005) and represents one of the foremost mammalian adaptations to life on a rotating planet. Photoperiodic information travels from the retina to the seat of the mammalian master clock in the suprachiasmatic nucleus (SCN) of the hypothalamus (Moore and Eichler, 1972; Stephan and Zucker, 1972). The molecular interplay of specialised ‘clock’ gene products within the SCN controls the circadian output to the pineal gland (Perreau-Lenz et al., 2004) and dictates the timing of MT production and release (Bartness et al., 1993). MT is synthesized and secreted solely during the dark period of the light/dark cycle and faithfully represents the duration of darkness, mirroring the seasonal changes in the length of day and night (Bartness and Goldman, 1989). Thus, this neuroendocrine pathway acts as a signal from the clock to the body conveying seasonal timing information to organs involved in reproduction and growth in both seasonal and non-seasonal breeding animals (Hansen, 1985; Bartness and Goldman, 1989; Morgan and Mercer, 1994).

Although not a seasonal breeder, MT production is strongly circadian in the cow (Berthelot et al., 1990) and many reproductive phenomena such as onset of puberty (Hansen et al., 1983; Kassim et al., 2008), return to cyclicity following parturition (Hansen and Hauser, 1983) and semen characteristics (Roussel et al., 1963, 1964) are influenced by photoperiod. However, MT also plays an important role in regulation of the somatotropic axis and the seasonal patterns of release of the important galactopoietic hormones prolactin and insulin like growth factor 1 (IGF-1) in cows (Dahl et al., 2000). A short duration of MT secretion stimulated by a long day photoperiod (LDPP) increases circulating prolactin and IGF-1 concentrations and these endocrine shifts are consistent with previously observed effects on lactation, body growth and carcass composition in cattle (Dahl et al., 2002). The specific mechanism responsible for greater circulating IGF-1 in cows exposed to longer photoperiod is unknown. However, there is growing evidence that circadian clocks may orchestrate the homeorhetic response to lactation, coordinating the changes in metabolism of multiple body tissues necessary to support this highly energy demanding process (Casey et al., 2009).

### 3. Discussion of Related Art

With increasing interest in the commercial applications of extending day length in cows for improved production efficiency has come an equal interest in determining the optimum light intensity for eliciting these effects. Lawson and Kennedy (2001) investigated the threshold levels of indoor housing white light intensity required to suppress MT to daytime levels in dairy heifers. Their findings suggested that less than 50 lux may be sufficient to prevent the initial nocturnal rise in plasma MT but that intensities exceeding 400 lux may be required to sustain this suppression for 8 hours on the first occasion of exposure (Lawson and Kennedy, 2001).

It is now known that blue, short wavelength light (465-485 nm), optimally stimulates a set of novel photoreceptors in the mammalian retina that regulate the SCN in the hypothalamus (Brainard et al., 2001; Thapan et al., 2001). These intrinsically photosensitive retinal ganglion cells (ipRGCs) contain a pigment called melanopsin (Provencio et al., 2000) and work in conjunction with photopigments in rods and cones to regulate biological rhythms (Berson et al., 2002; Hanifin and Brainard, 2007).

An aim is to identify the threshold level of blue light administered to a single eye in the dairy cow necessary to suppress MT to levels observed in well-lit housing. A further aim is to determine if supply of the identified threshold light intensity from a specialised headpiece that provides extended lighting to cows at pasture could effectively influence milk yield. Thirdly, we wish to explore additional potential applications of this technology where artificially extended lighting in indoor housing has previously been shown to impact growth, carcass composition, puberty onset and fertility in the bovine.

### SUMMARY OF THE INVENTION

According to a first aspect of the invention, there is provided a light-based method of causing a substantial suppression of melatonin production, the method comprising the step of shining blue light from an artificial blue light source into just one eye of a bovine, causing a substantial suppression of melatonin production in the bovine, sufficient to induce physiological change. An advantage is that a bovine may be kept outdoors while suppressing melatonin production, which reduces housing and feeding costs for the bovine. An advantage is that a bovine may be maintained solely outside on pasture while suppressing melatonin production. An advantage is that a bovine may be kept indoors with the lighting off while suppressing melatonin production, which reduces lighting costs for the bovine. An advantage is that the bovine may still have an unrestricted view from one eye, which means the bovine can still view its surroundings quite freely. An advantage is that various physiological changes in the bovine can be induced, such as increasing milk yield, improved feed efficiency, growth and higher average daily weight gains and improved fertility especially in relation to early puberty onset and reduced calving interval, by the suppression of melatonin production in the bovine. An advantage is that sustainability within the bovine food production industry is improved as resources can be better utilized by maintaining a bovine inside or outside while suppressing melatonin production.

The method may be one in which the physiological change is a non-therapeutic physiological change.

The method may be one comprising directing blue light into the bovine's eye for at least one selected period of time.

The method may be one including the step of providing to the bovine a combined total of natural and blue light from the artificial blue light source for at least approximately 16 hours during each 24 hour period. An advantage is more consistent physiological changes in the bovine can be induced, resulting from a more consistent exposure to blue light from day-to-day.

The method may be one including the step of keeping the bovine outdoors whilst the blue light from the artificial blue light source is shone into the just one eye of the bovine. An advantage is reduced housing and feeding costs for the bovine.

The method may be one in which the artificial light source produces only low intensity blue light. An advantage is reduced power consumption.

The method may be one in which the blue light has a peak wavelength of from 440 to 490 nm.

The method may be one in which the blue light has a peak wavelength of from 459 to 484 nm.

The method may be one in which the blue light has a peak wavelength of 465 nm.

The method may be one in which the artificial blue light source includes a white light source. The method may be one in which the white light source provides blue-enriched light eg. white light from LEDs that have a high blue component.

The method may be one in which the artificial blue light source includes a white light LED and a blue filter.

The method may be one in which the artificial blue light source comprises at least one blue LED. An advantage is efficient conversion of electrical energy into blue light.

The method may be one in which the artificial blue light source includes a white light LED and no blue filter.

The method may be one in which light incident on the just one eye of the bovine is blue.

The method may be one including the step of directing diffused blue light into the bovine's eye. An advantage may be reduced bovine discomfort due to eliminating a concentrated light source.

The method may be one in which the artificial light source provides a blue light intensity of from 10 to 400 lux at the just one eye of the bovine. For example, studies have shown that low intensity blue light as low as 10 lux can elicit an effect of causing a substantial suppression of melatonin production in the bovine, if preceded by high intensity blue light.

The method may be one in which the artificial light source provides a blue light intensity of from 100 to 300 lux at the just one eye of the bovine.

The method may be one in which the artificial light source provides a blue light intensity of from 200 to 250 lux at the just one eye of the bovine.

The method may be one in which the lux levels specified refer to the combined effect of all artificial light sources which are illuminating the bovine's eye.

The method may be one in which the artificial blue light source comprises more than one artificial light source (e.g. LEDs). An advantage is robustness against the failure of an individual light source.

The method may be one in which the blue light is directed into only a single one of the bovine's eyes at any given time. An advantage is that the bovine still has an unrestricted view from one eye, which means the bovine can still view its surroundings quite freely.

The method may be one including the step of shielding the light source to substantially avoid direct illumination of the bovine's eye. An advantage may be reduced bovine discomfort due to eliminating a concentrated light source.

The method may be one in which the bovine is a cow, a bull, a heifer, a steer, an ox, a calf, or a buffalo.

The method may be one wherein the bovine is a cow, and the physiological change includes increased milk yield from the cow.

The method may be one wherein the cow is a multiparous cow.

The method may be one wherein the increased milk yield is from 1% to 9%, or from 1% to 11%.

The method may be one wherein the cow is a primiparous cow at first lactation.

The method may be one wherein the bovine is a cow, and the physiological change includes one or more of faster onset of puberty, earlier first conception, or improved ovarian growth.

The method may be one wherein the physiological change includes an increased rise in circulating gonadotropins.

The method may be one wherein the bovine is a cow, and the physiological change includes increased frequency of estrus behavior, or improved mammary development.

The method may be one wherein the physiological change includes increased body weight at puberty, or improved feed efficiency, or greater body growth and total weight gain, or increased heart girth, or increased average daily gain (ADG).

The method may be one wherein the bovine is a heifer, and the physiological change includes increased growth of a pre-pubertal heifer with an initial low body weight, or a heavier heifer at parturition, or a taller heifer at parturition.

The method may be one wherein the physiological change includes increased carcass protein content, or reduction in carcass subcutaneous fat.

The method may be one wherein the bovine is a cow, and the physiological change includes faster return to reproductive competence post-partum, or increased conception rates and reduced artificial inseminations per animal, or reduction in calving interval, or increases in 60-day non-return rate, or younger first calvers, or reduced risk of twin pregnancies.

The method may be one wherein the bovine is a bull, and the physiological change includes slowed decline in semen viability and concentration as a result of high temperature and humidity, or improved semen quality.

According to a second aspect of the invention, there is provided a device for causing a substantial suppression of melatonin production, the device comprising a headpiece fittable to a bovine's head, and including an artificial blue light source, the headpiece operable to shine blue light from the artificial blue light source into an eye of the bovine, causing a substantial suppression of melatonin production in the bovine, sufficient to induce physiological change. An advantage is that a bovine may be kept outdoors while suppressing melatonin production, which reduces housing costs for the bovine. An advantage is that a bovine may be kept indoors with the lighting off while suppressing melatonin production, which reduces lighting costs for the bovine. An advantage is that various physiological changes in the bovine can be induced, such as increasing milk yield, improved feed efficiency, growth and higher average daily weight gains and improved fertility especially in relation to early puberty onset and reduced calving interval, by the suppression of melatonin production in the bovine.

The device may be one wherein the headpiece is operable to shine blue light from the artificial blue light source into just one eye of the bovine. An advantage is that the bovine still has an unrestricted view from one eye, which means the bovine can still view its surroundings quite freely.

The device may be one wherein the device is configured to direct blue light into the bovine's eye for at least one selected period of time.

The device may be one wherein the device is configured to provide to the bovine a combined total of natural and blue light from the artificial blue light source for at least approximately 16 hours during each 24 hour period. An advantage is more consistent physiological changes in the bovine can be induced, resulting from a more consistent exposure to blue light from day-to-day.

The device may be one wherein the device is one in which the artificial light source produces only low intensity blue light.

The device may be one wherein the blue light has a peak wavelength of from 440 to 490 nm.

The device may be one wherein the blue light has a peak wavelength of from 459 to 484 nm.

The device may be one wherein the blue light has a peak wavelength of 465 nm.

The device may be one wherein the artificial blue light source includes a white light source. The device may be one in which the white light source provides blue-enriched light eg. white light from LEDs that have a high blue component.

The device may be one wherein the artificial blue light source includes a white light LED and a blue filter.

The device may be one wherein the artificial blue light source comprises at least one blue LED.

The device may be one wherein the artificial blue light source includes a white light LED and no blue filter.

The device may be one wherein the device is configured such that light incident on the just one eye of the bovine is blue.

The device may be one wherein the device includes a diffuser arranged to diffuse blue light, wherein the diffused blue light is directed into the bovine's eye. An advantage may be reduced bovine discomfort due to eliminating a concentrated light source.

The device may be one wherein the artificial light source provides a blue light intensity of from 10 to 400 lux at the just one eye of the bovine.

The device may be one wherein the artificial light source provides a blue light intensity of from 100 to 300 lux at the just one eye of the bovine.

The device may be one wherein the artificial light source provides a blue light intensity of from 200 to 250 lux at the just one eye of the bovine.

The device may be one wherein the lux levels specified refer to the combined effect of all artificial light sources which illuminate the bovine's eye.

The device may be one wherein the artificial blue light source comprises more than one artificial light source (e.g. LEDs). An advantage is robustness against the failure of an individual light source.

The device may be one wherein the device is arranged such that the blue light is directed into only a single one of the bovine's eyes at any given time.

The device may be one wherein the light source is shielded to substantially avoid direct illumination of the bovine's eye.

The device may be one wherein the bovine is a cow, a bull, a heifer, a steer, a calf, an ox, or a buffalo.

The device may be one wherein the headpiece comprises a mask.

The device may be one wherein the device includes a control that is capable of turning on the device such that when turned on, the control leaves the device on so that the bovine receives a combined total of natural and blue light from the artificial blue light source for approximately 16 hours during each 24 hour period. An advantage is more consistent physiological changes in the bovine can be induced, resulting from a more consistent exposure to blue light from day-to-day.

The device may be one wherein the device includes a blinker that covers the eye into which blue light from the artificial blue light source is shone.

The device may be one wherein the device is weatherproof.

The device may be one wherein the device is waterproof.

The device may be one wherein the headpiece contains only a single light source for directing blue light into only a single eye of the bovine.

The device may be one wherein the headpiece includes an automatic timer for turning the light source on and off at selected times.

The device may be one wherein the headpiece comprises a fabric base, the fabric base including eye openings and fasteners for fastening the headpiece around the bovine's head.

The device may be one wherein the headpiece includes a power supply for the light source.

The device may include a battery to power the power supply, and may further include solar cell apparatus to charge the battery using solar power, or may further include a kinetic energy charger apparatus to charge the battery e.g. to charge the battery using movement of the bovine.

According to a third aspect of the invention, there is provided use of the device of any aspect according to the second aspect of the invention, (i) wherein the bovine is a cow, and the physiological change includes increased milk yield from the cow; or

(ii) wherein the bovine is a cow, and the physiological change includes one or more of faster onset of puberty, earlier first conception, or improved ovarian growth; or
(iii) the physiological change includes an increased rise in circulating gonadotropins; or
(iv) wherein the bovine is a cow, and the physiological change includes increased frequency of estrus behavior, or improved mammary development; or
(v) the physiological change includes increased body weights at puberty, or improved feed efficiency, or greater body growth and total weight gain, or increased heart girth, or increased average daily gain (ADG); or
(vi) wherein the bovine is a heifer, and the physiological change includes increases in growth of a pre-pubertal heifer with an initial low body weight, or a heavier heifer at parturition, or a taller heifer at parturition; or
(vii) the physiological change includes increased carcass protein content, or reduction in carcass subcutaneous fat; or
(viii) wherein the bovine is a cow, and the physiological change includes faster return to reproductive competence post-partum, or increased conception rates and reduced artificial inseminations per animal, or reduction in calving interval, or increases in 60-day non-return rate, or younger first calvers, or reduced risk of twin pregnancies; or
(ix) wherein the bovine is a bull, and the physiological change includes slowed decline in semen viability and concentration as a result of high temperature and humidity, or improved semen quality.

According to a fourth aspect of the invention, there is provided a system including a device for causing a substantial suppression of melatonin production, the device comprising a headpiece fittable to a bovine's head, and including an artificial blue light source, the headpiece operable to shine blue light from the artificial blue light source into an eye of the bovine, causing a substantial suppression of melatonin production sufficient to induce physiological change, in which the light source is operable to provide blue light at the eye of the bovine, the system including a control in connection with the device, in which the control is configured to turn on the device, wherein the device is configured to direct blue light into the bovine's eye for at least one selected period of time.

The system may be one wherein the device is configured to provide the bovine with a combined total of natural and blue light from the artificial blue light source for approximately 16 hours during each 24 hour period.

The system may be one wherein the headpiece is operable to shine blue light from the artificial blue light source into just one eye of the bovine, in which the light source is operable to provide blue light at the just one eye of the bovine.

The system may be one wherein the connection is a wired connection.

The system may be one wherein the connection is a wireless connection.

The system may be one wherein the device is a device of any aspect of the second aspect of the invention.

According to a fifth aspect of the invention, there is provided use of the system of any aspect of the fourth aspect of the invention, (i) wherein the bovine is a cow, and the physiological change includes increased milk yield from the cow; or

(ii) wherein the bovine is a cow, and the physiological change includes one or more of faster onset of puberty, earlier first conception, or improved ovarian growth; or
(iii) the physiological change includes an increased rise in circulating gonadotropins; or
(iv) wherein the bovine is a cow, and the physiological change includes increased frequency of estrus behavior, or improved mammary development; or
(v) the physiological change includes increased body weights at puberty, or improved feed efficiency, or greater body growth and total weight gain, or increased heart girth, or increased average daily gain (ADG); or
(vi) wherein the bovine is a heifer, and the physiological change includes increases in growth of a pre-pubertal heifer with an initial low body weight, or a heavier heifer at parturition, or a taller heifer at parturition; or
(vii) the physiological change includes increased carcass protein content, or reduction in carcass subcutaneous fat; or
(viii) wherein the bovine is a cow, and the physiological change includes faster return to reproductive competence post-partum, or increased conception rates and reduced artificial inseminations per animal, or reduction in calving interval, or increases in 60-day non-return rate, or younger first calvers, or reduced risk of twin pregnancies; or
(ix) wherein the bovine is a bull, and the physiological change includes slowed decline in semen viability and concentration as a result of high temperature and humidity, or improved semen quality.

There is provided a device for causing a substantial suppression of melatonin production, the device comprising a headpiece fittable to a bovine's head, and including one or more artificial blue light sources, the headpiece operable to shine blue light from the one or more artificial blue light sources into the eyes of the bovine, causing a substantial suppression of melatonin production in the bovine, sufficient to induce physiological change. The device may include any aspect according to the second aspect of the invention.

The advantages of the invention include that mobile timed lighting can be provided to bovines while they remain in their natural outdoor pasture environment. This means that the bovines do not need to be housed indoors with the associated bedding, feed, electricity and labour costs. Alternatively, the advantages of the invention include that mobile timed lighting can be provided to bovines while they remain indoors but without indoor lighting. This saves on indoor lighting costs.

In this document, the season implied by a stated month is that implied for the northern hemisphere, unless stated otherwise. For the southern hemisphere, for the same seasonal effect to be obtained as in the northern hemisphere, the number of the month should be shifted by six, to obtain the same effect, as would be clear to one skilled in the art. For example, January in the northern hemisphere is seasonally equivalent to July in the southern hemisphere.

### BRIEF DESCRIPTION OF THE FIGURES

Aspects of the invention will now be described, by way of example(s), with reference to the following Figures.

FIG. 1. Summary of ten published papers investigating the effect of extended daylength on milk yield in lactating cows. Full citations appear in the references section. Black bars indicate the average daily milk yield (kg/d) of cows under natural photoperiod (range of 8 to 13.5 h light/d; control) and white bars indicate milk yield (kg/d) of cows exposed to extended photoperiod of 16 to 18 h of light/d. Adapted from Dahl, G. E. and Petitclerk, D (2003), ‘Management of photoperiod in the dairy herd for improved production and health’.

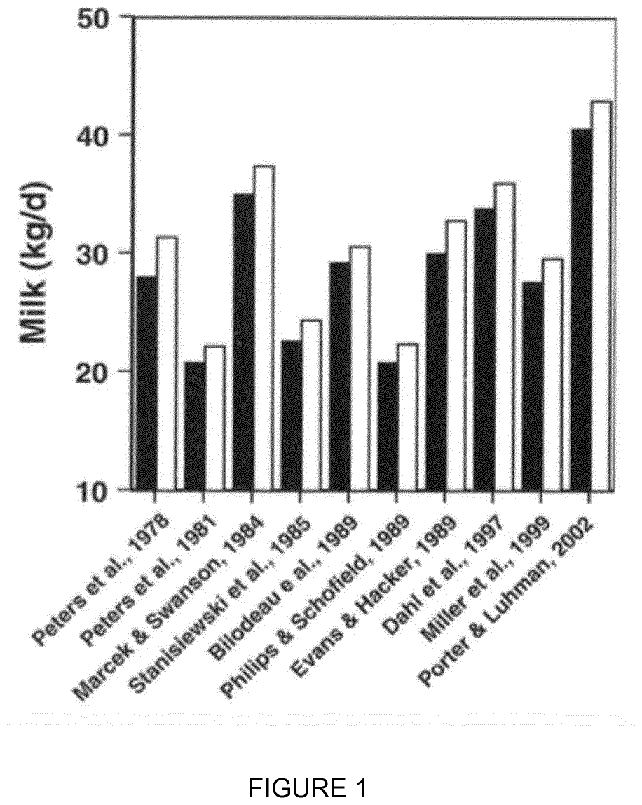

FIG. 2. Schematic outline of Study 1 schedule for an individual cow. During the experimental period, cows were assigned different masks on different nights. Each cow received each treatment light intensity (0 lux; 70 lux; 125 lux; 175 lux and 225 lux) in a 5×5 Latin Square design followed by a night of LIGHTS ON (>200 lux). See text for details.

FIG. 3. Mean plasma MT levels (pg/mL) from all animals at time points 16:00 to 00:00 on all treatment nights and on the LIGHTS ON night. A dose dependent suppression of MT by increasing light intensities is observed (P<0.0001). Only the 225 lux treatment did not differ from LIGHTS ON values (P>0.05). Data are presented as means±SEM.

FIG. 4. Changes in mean plasma MT levels over time. Baseline blood samples were taken just prior to 16:00 and before lights off. Different light treatment intensities were administered to a single eye via head worn light masks and are presented as different lines and symbols, according to the key. The zero lux line, and the 225 lux line, are also individually indicated, for clarity. LIGHTS ON is 237±68 lux. Following the sample at 00:00, light masks were removed or the lights were turned off, for the LIGHTS ON night. Data are presented as means±SEM.

FIG. 5. Mean plasma MT levels at 18:00 and 22:00 on break nights. MT values were consistently low at 18:00 (4.94±1.71 pg/ml) and high at 22:00 (20.66±1.71 pg/ml) indicating that there were no carryover effects on MT production from the previous night's treatment. Data are presented as box and whisker plots with error bars representing minimum and maximum values.

FIG. 6. (A) Mean daily milk yield (kg/day) of Light treated and Control groups from Week 1 to Week 12 of lactation. A significant effect of treatment was observed (P<0.0001) representing an increase yield in the Light group of 0.99 kg/day, or 4.3%. (B) A significant treatment*parity interaction (P<0.0001) permitted separate analysis of productivity from primiparous and multiparous cows. No effect of treatment was observed for primiparous cows (P>0.05), whereas a significant effect of treatment was observed in multiparous cows (P<0.0001). This effect of Light treatment represented a mean average increased yield of 2.25 kg/day or an 8.6% production increase. Data are presented as means±SEM.

FIG. 7. Image of a dairy cow from the Light treated group in Study 2 wearing an example of a light mask, at pasture.

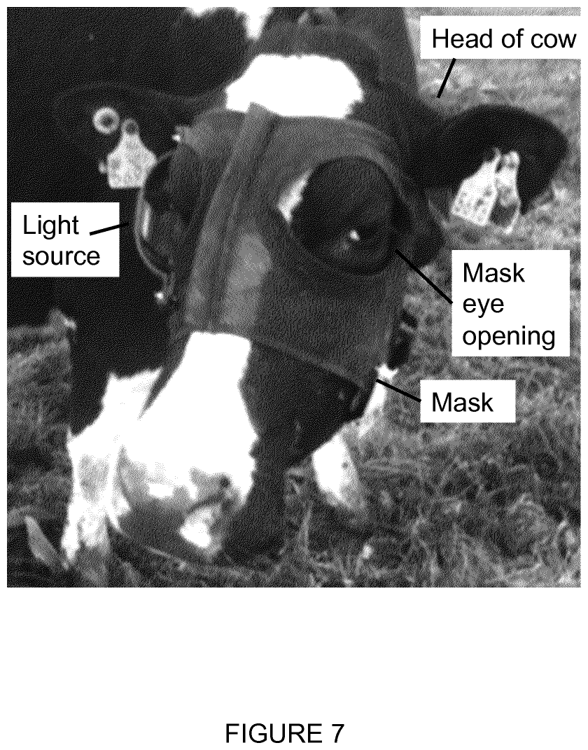

### DETAILED DESCRIPTION

In an example, there is provided a light-based method of causing a substantial suppression of melatonin production, the method comprising the step of shining blue light from an artificial blue light source into an eye (e.g. just one eye) of a bovine, causing a substantial suppression of melatonin production sufficient to induce physiological change. The physiological change may be a non-therapeutic physiological change. Light incident on the eye (e.g. just one eye) of the bovine may be blue. The light source may be a white light source. White light includes blue light within the white light.

The method may include the step of providing to the bovine a combined total of natural and blue light from the artificial blue light source for at least approximately 16 hours during each 24 hour period.

The method may include the step of keeping the bovine outdoors whilst the blue light from the artificial blue light source is shone in an eye (e.g. just one eye) of the bovine.

The method of may be one in which the artificial light source produces only low intensity blue light.

In an example, there is provided a device for causing a substantial suppression of melatonin production, the device comprising a headpiece fittable to a bovine's head, and including an artificial blue light source, the headpiece operable to shine blue light from the artificial blue light source into an eye (e.g. just one eye) of the bovine, causing a substantial suppression of melatonin production sufficient to induce physiological change, in which the light source is operable to provide blue light at the eye (e.g. just one eye) of the bovine. The headpiece may comprise a mask. Light incident on the eye (e.g. just one eye) of the bovine may be blue. The light source may be a white light source.

The device may include a control that is capable of turning on the device such that when turned on, the control leaves the device on so that the bovine receives a combined total of natural and blue light from the artificial blue light source for approximately 16 hours during each 24 hour period.

The device may include a blinker that covers the eye into which blue light from the artificial blue light source is shone. The device may be weatherproof. The device may be waterproof.

In an example, there is provided a system including a device for causing a substantial suppression of melatonin production, the device comprising a headpiece fittable to a bovine's head, and including an artificial blue light source, the headpiece operable to shine blue light from the artificial blue light source into an eye (e.g. just one eye) of the bovine, causing a substantial suppression of melatonin production sufficient to induce physiological change, in which the light source is operable to provide blue light at the eye (e.g. just one eye) of the bovine, the system including a control in connection with the device, in which the control is configured to turn on the device, to provide the bovine with a combined total of natural and blue light from the artificial blue light source for approximately 16 hours during each 24 hour period. The headpiece may comprise a mask. Light incident on the eye (e.g. just one eye) of the bovine may be blue. A connection may be a wired connection. A connection may be a wireless connection. The light source may be a white light source.

Preferably the blue light has a peak wavelength of from 440 to 490 nm, preferably 459 to 484 nm, and most preferably 465 nm. The artificial light source may produce only low intensity blue light. The artificial blue light source may include a white light LED and a blue filter. The artificial blue light source may comprise at least one blue LED. In an alternative, a light source may be a white light LED with no blue filter.

Preferably the artificial light source has an intensity of from 10 to 400 lux, more preferably from 100 to 300 lux most preferably from 200 to 250 lux. The artificial blue light source may comprise more than one artificial light source (e.g. LEDs) and the lux levels specified above refer to the combined effect of all artificial light sources which are illuminating the bovine's eye.

In contrast, natural daylight may provide anywhere from 1000 to 10,000 lux of light, depending on cloud cover.

Preferably the mask contains only a single light source for directing blue light into only a single eye of the bovine.

The apparatus preferably includes an automatic timer for turning the light source on and off at selected times.

There is further provided a method of suppressing melatonin synthesis in a bovine, comprising directing diffused blue light into the bovine's eye for at least one selected period of time.

Preferably the blue light is directed into only a single one of the bovine's eyes at any given time.

There is provided an apparatus for suppressing melatonin synthesis in a bovine, comprising a blinker having an internal surface of which at least a part is diffusing, and a source of light positioned for direction into the bovine's eye via the diffusing surface, the light source and diffusing surface being configured such that the light directed into the bovine's eye is blue. Preferably the diffusing surface of the blinker is diffusively reflective. The blinker may be mounted in a headpiece (e.g. a mask) adapted for fitting to a bovine's head. The headpiece (e.g. mask) contains only a single blinker for directing blue light into only a single eye of the bovine. The apparatus may include an automatic timer for turning the light source on and off at selected times.

The light source may be shielded to substantially avoid direct illumination of the bovine's eye.

It will be understood that the internal surface of the blinker is the surface of the blinker which in use faces the bovine's eye and is usually, but not necessarily, concave or approximately concave.

In an example, the light source emits blue light and the diffusing surface does not significantly filter the blue light.

The headpiece (e.g. mask) may comprise a fabric base, having eye openings and fasteners for fastening the headpiece (e.g. mask) around the bovine's head.

A light source unit (eg. a blinker) including a blue LED, and optionally a reflecting surface, may be fitted to only one of the two eye openings in the headpiece, since we have found that melatonin can be suppressed by administration of light to a single bovine eye. This is important since the blue light administered to a single bovine eye will not impede the natural behaviour of bovines maintained outdoors at night, whereas light administered to both eyes might impede vision and movement. Bovines that are blind in a single eye can move and behave normally. The other eye opening in the headpiece would normally be left open. It would also be possible to swap the light source unit (eg. a blinker) from one eye opening to the other eye opening at various times, to balance the effect on the bovine, as long as the blue light is only administered to one eye at any given time.

The headpiece (e.g. mask) may include a power supply for the light source unit (eg. in a pocket of the headpiece). The power supply is connected to the light source (eg. a LED) by leads and has a battery compartment for a replaceable and/or rechargeable battery. The power supply may include an on/off switch. In an example, it is possible to operate the headpiece device manually, by switching the power supply on and off at various times, but in another example the power supply may include an automatic timer to turn the light source (eg. a LED) on and off at pre-selected times. Such a timer may be a manually settable timer or, more preferably, a programmable electronic circuit which can be programmed by computer, e.g. via a USB port included in the headpiece, or via a wireless interface in the headpiece. In an example, the power supply has approximate dimensions 96 mm×60 mm×26 mm. Alternative power supplies include those charged by solar power using a solar cell, or including a kinetic energy charger e.g. charged using movement of the bovine.

In an example, a device may include an automatic ambient light sensor that, in combination with a timer, can modulate the light source turn-on and/or turn-off times for maximum efficiency.

A bovine may be a cow, a bull, a heifer, a steer, a calf, an ox, or a buffalo, for example.

### Methods: Study 1

### Animals and Experimental Protocol

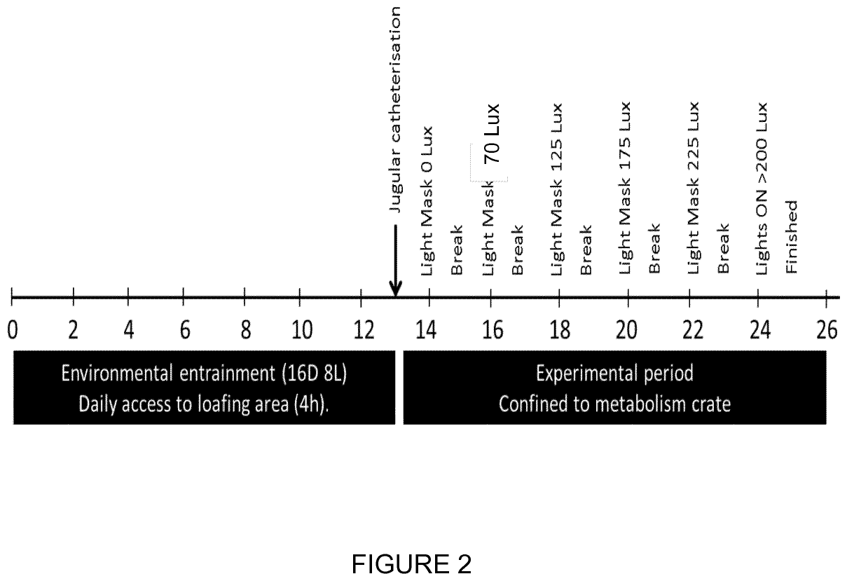

In November 2015, five healthy, non-lactating Holstein-Friesian cows were exposed to a 14-day environmental conditioning period that comprised 8 hours of light, 16 hours of dark (8L:16D), where mean (±SD) white light intensity levels by day (08:00-16:00) at eye level were 237±68 lux. During this entrainment period, walking and socialisation was facilitated by moving the cows from the metabolism shed, where they were housed in individual stalls, to a loose-housed straw-bedded pen with access to natural daylight for 4 hours each day (8.30 am to 12.30 pm). Environmental lighting in the loafing area was also measured daily. On the fourteenth day of the entrainment period, indwelling jugular catheters were fitted using sterile technique, and the animals were confined to the metabolism shed for 12 consecutive days thereafter. This ensured complete elimination of external environmental influences on MT secretion. The metabolism chamber was light-proofed such that at lights-off (16:00) the ambient light levels dropped to <1 lux and further husbandry procedures and sample collection was conducted using dim red light from head-mounted or handheld flashlights. Beginning on Day 14, each cow was exposed to each of the following light intensities for 8 additional hours (16:00-00:00) using a 5×5 Latin Square design; <1 lux; 70 lux; 125 lux; 175 lux and 225 lux (FIG. 2). Each treatment night was followed by a break night, where the animals again received 8L:16D. Two days after completion of the different light intensity treatments, all cows were exposed to the indoor lighting system until 00:00 (LIGHTS ON). Each light treatment was administered using an LED lighting system and electronic platform that provides short wavelength blue light (468 nm) incorporated into a custom designed headpiece to fit a dairy cow. Prototype bovine light masks were designed to fit comfortably according to the specific dimensions of the cow's head conformation. The <1 lux treatment was provided by fitting a mask without functional LEDs.

### Blood Sampling

A heparinised (200 units mL⁻¹) blood sample was collected from each animal before lights off at 16:00, and was followed by samples collected at 17:00, 18:00, 20:00, 23:00, 00:00 and 01:00. On break nights, blood samples were collected at 18:00 and 22:00. This was to verify that there were no carryover effects of the previous night's treatment on MT levels. Plasma was harvested from all samples and stored at −20° Celsius until assayed for MT using a commercially available RIA.

### MT Radioimmunoassay (RIA)

MT was measured using a Bühlmann MT RIA kit (RK-MEL2, ALP-CO Diagnostics). Serum aliquots (500 μL) were column-extracted using a vacuum manifold (Visiprep-DL Solid Phase Extraction Vacuum Manifold) according to the directions of the manufacturer and reconstituted in 500 μL of incubation buffer solution provided with the kit. Aliquots of the reconstituted extracted samples (200 μL) were assayed in duplicate in a single assay. The inter and intra-assay coefficients of variance for low and high quality controls were 10.8% and 12.9%; and 6.2% and 4.7%, respectively. As documented by the manufacturer, the efficiency of the extraction method was >90%, while the assay had an estimated functional sensitivity of 0.9 pg/mL (coefficient of variance−10%) and an estimated analytical sensitivity of 0.3 pg/mL. This assay has been used previously to examine MT levels in bovine plasma serum (Lawson and Kennedy, 2001).

### Data Analysis

MT data were log transformed and analysed using SAS (SAS System, Inc., Cary, N.C., USA) mixed models with treatment, period, hour and treatment×hour as fixed effects and cow as a random effect. MT data from four of the break nights were analysed using two-way repeated measures analysis of variance (ANOVA) followed by Sidak's multiple comparisons post-hoc tests using GraphPad Prism 6 for Mac OS X. A P-value of <0.05 was considered significant. Data are presented as means±SEM (standard error of the mean).

### Methods: Study 2

### Animals and Experimental Protocol

Forty spring-calving Holstein-Friesian cows were blocked for parity, calving date and Economic Breeding Index for Milk Production (EBIMilk) and randomly assigned to either a CONTROL treatment or blue light to a single eye (LIGHT) treatment from day of calving through 12 weeks of lactation (February-May 2017). The CONTROL group consisted of 6 primiparous and 12 multiparous cows and the LIGHT group consisted of 6 primiparous and 13 multiparous animals. The LIGHT group were fitted on day of calving with light masks providing 225 lux of blue light to the right eye that activated on a timer from 17:00 until 00:00 daily (16L:8D-18L:6D, as season progressed). Cows were milked twice daily at 07:30 and 15:00, and milk yield (kg) was recorded daily at the morning and evening milkings using electronic milk meters (Dairy Master, Causeway, Co. Kerry, Ireland). An average daily milk yield (kg) was computed for each cow on each week of lactation. Light masks were checked for positioning and the lens of the light source cleaned biweekly.

### Data Analysis

Mean daily milk yield (kg/d) was analysed using SAS (SAS System, Inc., Cary, N.C., USA) mixed models with treatment, week of lactation, parity and treatment×week of lactation and treatment×parity as fixed effects and cow as a random effect. A P-value of <0.05 was considered significant. Data are presented as means±SEM.

### Results

### Study 1

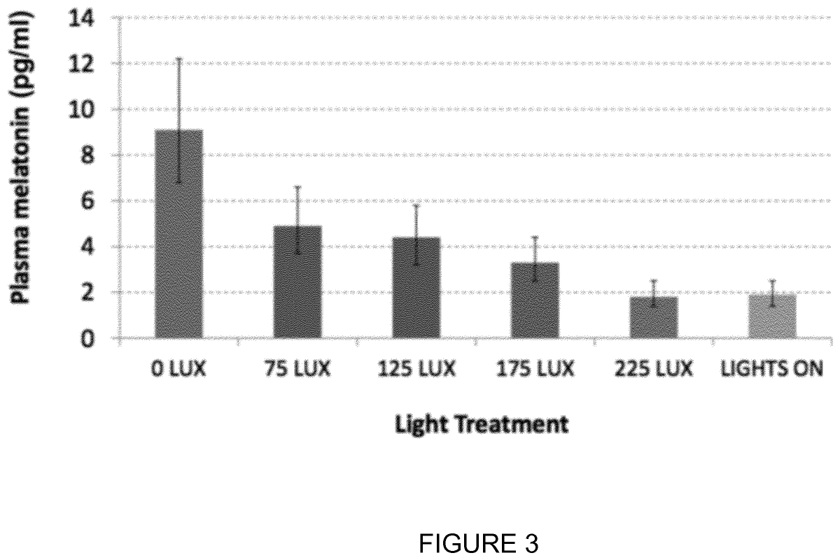

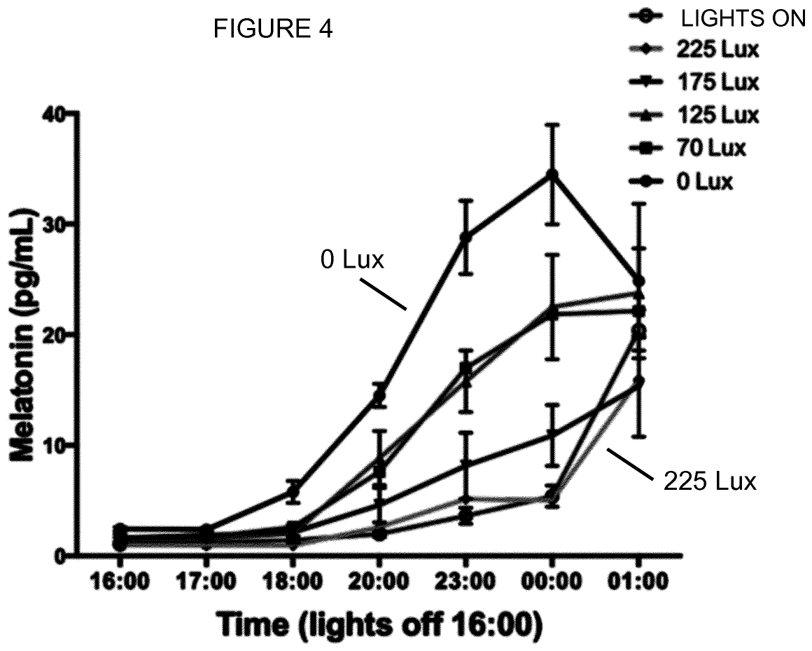

A dose-dependent effect of treatment on mean circulating MT concentrations between 16:00 and 00:00 was observed (P<0.0001; FIG. 3). Mean plasma MT concentration was low and did not differ (P>0.05) among treatments at 16:00, when the cows were still exposed to the final minutes of the basic 8 h photoperiod (237±68 lux), or at 17:00 and 18:00 (P>0.05, respectively). At 20:00, there was no difference (P>0.05) between 0 lux and the 70 lux or 125 lux treatment; however, 175 lux, 225 lux and LIGHTS ON treatments had lowered (P<0.05, P<0.01, P<0.001; respectively) plasma MT, compared to 0 lux. At 23:00 and 00:00, all treatments had lowered plasma MT compared to 0 lux (P<0.01). Only the 225 lux treatment did not differ from LIGHTS ON plasma MT levels at any sampling time (P>0.05). At 01:00, representing one hour following lights off and/or removal of treatment light masks there was no difference between any treatments for plasma MT (P>0.05). Data for all time points and treatments are graphically presented in FIG. 4.

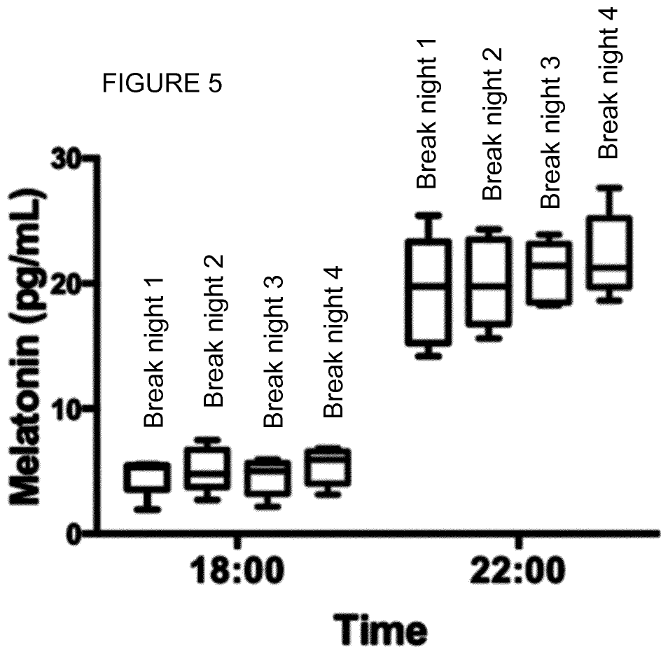

On break nights, there was a main effect of time (P<0.0001) but no effect of night (P>0.05) on plasma MT levels. At 18:00, mean levels were consistently low (4.67, 5.14, 4.54 and 5.41 ng/mL) and at 22:00 levels were consistently higher (19.38, 20.07, 20.95, 22.22 ng/mL). These data confirm that there were no carryover effects of the previous night's treatment on subsequent MT levels the following night. Data are graphically presented in FIG. 5.

### Study 2

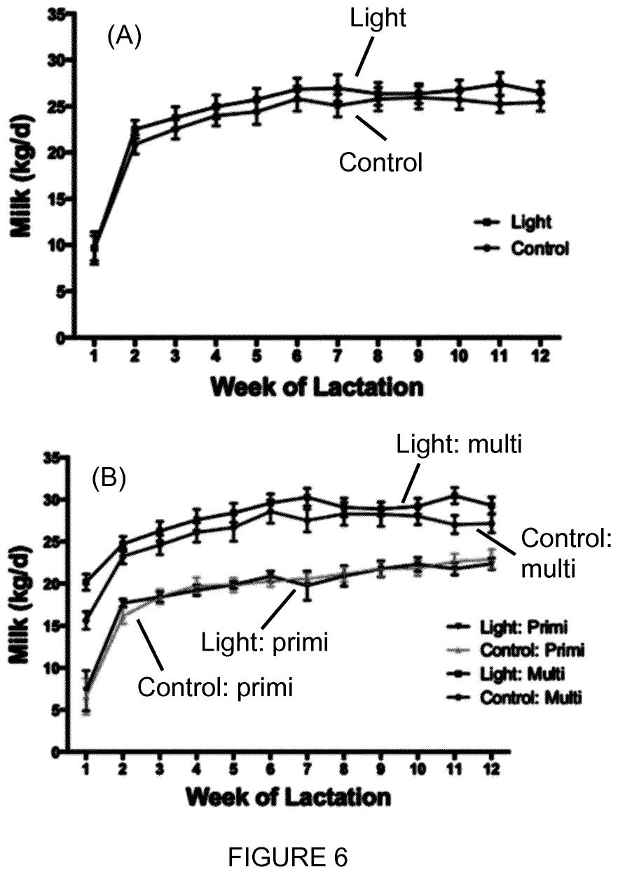

Of the initial 20 cows in the CONTROL group, two animals were removed from the study due to ill-health. A single animal was removed from the LIGHT group due to being discovered to be not pregnant. During the trial, light masks were replaced frequently when the light source was damaged or had become detached from the headpiece. Frequent cleaning of the light source was required to remove built up faecal matter and debris. Milk yield analysis for the remaining 37 cows revealed that there was a main effect of treatment (P<0.0001) and a main effect of week of lactation (P<0.0001), whereby animals in the LIGHT group had a mean increased milk production of 0.99 kg/d. There was also a treatment×parity interaction (P<0.0001). Groups were then sub-divided into primiparous and multiparous animals for further analysis. There was a main effect of treatment (P<0.0001) for multiparous cows in the LIGHT group with a mean increase of 2.25 kg/d of milk. There was no effect (P>0.05) of treatment on primiparous cows with an observed mean decrease of 0.27 kg/day. Data for all animals and for the breakdown between primiparous and multiparous animals are presented in FIGS. 6 (A) and (B), respectively.

### DISCUSSION

Previous investigations of lighting regimes to elicit LDPP responses in cows have included the use of fluorescent, metal halide, and high pressure sodium (HPS) lights (Peters et al., 1978; Kennedy et al., 2004; Rius and Dahl, 2006) as well as the more recent introduction of LEDs (Harner and Zulovich, 2014) and light intensities ranging from 36 to 400+ lux measured at eye level (Lawson and Kennedy, 2001). Without exception, these regimes for photoperiodic management require indoor housing of animals, with associated high feeding, management and energy costs (Lawson and Kennedy, 2001; Fariña et al., 2011) and dense stocking rates where animal welfare issues arise (Marcek and Swanson, 1983). Without exception, the primary physiological effect desired by these lighting regimes is suppression of levels of pineal MT production (Stanisiewski et al., 1988; Muthuramalingam et al., 2006; Kassim et al., 2008). Here we identify for the first time the minimum blue light intensity that effectively suppresses the hormone MT to levels similar to those observed in well-lit housing when administered to a single eye, as 225 lux.

Importantly, study 1 reports a dose-dependent response on MT suppression by different light intensities provided by head-worn light masks following a single exposure to a light stimulus. MT is one of the most stable outputs from the mammalian circadian system (Benloucif et al., 2005) and has been shown to display highly circadian patterns of production in the bovine (Berthelot et al., 1990). By definition, a circadian rhythm can only adapt gradually to changes in the light-dark cycle. Artificial time cues associated with new lighting regimes pose a challenge to a circadian system evolved to deal with gradual seasonal changes in day length provided by the natural environmental light-dark cycles. Time is required to allow readjustment of molecular and cellular events that occur both in the SCN and peripheral tissues during resetting to a new 24 h cycle (Yamazaki et al., 2000; Reddy et al., 2002). However, in contrast to the desynchronization between the internal clock and the external environment that is experienced during transmeridian travel or shift work (Gander et al., 1985; Loat and Rhodes, 1989), where both the timing of dawn and dusk are displaced, initiating an LDPP regime only requires the circadian SCN pacemaker to respond to an extension in duration of daylight. Specific clock gene components of the molecular oscillator in the SCN are rapidly induced by light during early night (Shearman et al., 1997; Yan et al., 1999; Miyake et al., 2000), potentially explaining how the circadian system resets more readily when exposed to the lengthened hours of afternoon light associated with westward transmeridian travel compared to eastward travel (Yamazaki et al., 2000; Reddy et al., 2002). Nevertheless, it is expected that resynchronisation time to an LDPP would take days, if not weeks, especially in an animal where MT production is strongly circadian (Berthelot et al., 1990).

For these reasons, we hypothesize that MT suppression to daytime levels would be attained following repeated exposure to lower light intensities delivered to one eye and within the blue light spectrum. This hypothesis is supported by studies showing that repeated daily photoperiod extension using white light intensities between 114 and 231 lux improved cattle productivity (Peters et al., 1978, 1981). Furthermore, Resken et al. (1999) suggested that repeated daily exposure to low levels of light can have a positive effect on milk yield following the observation that photoperiod extension with dim illumination (mean 36 lux, range 4 to 160 lux) increased milk production in dairy cattle. Importantly, the authors noted that this response to dim light occurred when preceded by high intensity light during the day (Reksen et al., 1999), as would be experienced by grazing cattle.

In a previous study examining MT suppression by white light, Lawson and Kennedy (2001) observed that only a 400 lux light intensity could come closest to suppressing MT to the low levels observed at baseline values by day. In study 1, we achieve equal suppression levels with 225 lux short-wavelength blue light to levels observed under LIGHTS ON. This is doubly impressive given the administration of blue light to a single eye versus exposure of ambient light to both eyes in the previous study (Lawson and Kennedy, 2001). This difference is likely explained by the wavelengths of light used. Melanopsin, the photopigment identified in ipRGCs (Provencio et al., 2000) that are primarily responsible for signalling photic information to the SCN and thus regulating MT, shows a peak spectral sensitivity for short-wavelength blue light (Brainard et al., 2001; Thapan et al., 2001) within the range employed in the cow light mask described here. Furthermore, it was recently discovered that stimulation by light of a single melanop sin containing ipRGC within the retina can bilaterally innervate the SCN (Fernandez et al., 2016). This helps explain why blue light administration to a single eye can have a strong suppressive effect on MT production.

Further evidence for the circadian nature of MT production in the cow is the 2 h delay in its nocturnal rise in study 1 under 0 lux where cows experienced darkness from 16:00. This contrasts with Lawson and Kennedy (2001) who observed a significantly increased MT level in the first 2.5 hours under 0 lux. This slight variation in results may be attributed to differences in the sampling intervals between studies or to the length of the pre-experiment environmental conditioning period. The previous study pooled samples taken at 20 min intervals from 16:40 to 18:40 whereas we collected a single sample at 16:00, 17:00 and 18:00. It is possible that the pooled samples may have had a mean that was skewed towards the lower MT levels observed up until 18:00 in study 1. Lawson and Kennedy (2001) also exposed animals to a 21-day light conditioning period whereas only a 14-day period was utilised in study 1. Additionally, it is worth noting the differences in light intensities used in the animal housing between studies. Previously, it was reported that light intensities of 400 lux were provided from 08:00 to 16:00, however, no mean readings at eye level were described (Lawson and Kennedy, 2001). If the intensity at eye level was indeed 400 lux, then it was higher than the light intensity recorded at eye level in study 1 (237±68 lux) and may have elicited a more rapid nocturnal rise in MT following release into darkness.

An important finding in study 1 was the lack of any observed carryover effects of the previous night's light treatment on MT patterns the subsequent night. On each break night, MT levels were shown to be consistently lower at 18:00 and high at 22:00. An alternative finding might have confounded results. This again underlines how regulation of MT requires repeated exposure to a light stimulus to elicit a gradual adaptation to a new environmental signal relating to day length.

The gradual adaptation of the MT response to LDPP is supported by the findings that it takes two weeks on average to observe a rise in circulating IGF-1 in response to the initiation of an extended lighting programme (Dahl et al., 1997). Therefore, it is likely that for stable entrainment of the MT rhythm to occur, an approximate two-week timeframe is required in which MT patterns will likely respond to repeated exposure to a range of light intensities. Future studies should consider initiating LDPP two weeks in advance of calving due date to potentially elicit a faster effect on increasing milk yield post-partum. It is interesting that there was no perceivable difference in suppression levels attained by 70 lux and 125 lux in the current study. Regardless, we clearly demonstrate that to achieve suppression levels similar to those observed under an initial exposure to well-lit indoor housing conditions, an intensity of 225 lux short-wavelength blue light administered to one eye is required.

In study 2, we apply our findings from study 1 to a field trial investigating the influence of LDPP provided by a mobile light mask delivering 225 lux blue light to one eye on milk yield in dairy cows. Our results show for the first time that mobile lighting stimulates increased milk production in lactating dairy cows maintained at pasture. Our experimental groups were balanced for parity, with equal proportions of primiparous and multiparous cows. The milk yield difference across the entire group represented a 4.3% increase, or approximately 1 kg/d. When primiparous and multiparous animals were analysed separately, it became clear that LDPP had no effect on milk production in primiparous animals but that the difference observed in multiparous animals represented an 8.6% increase, or 2.25 kg/d. These results support the myriad studies highlighting the impact of LDPP on milk production in cows (Dahl et al., 2000).

Furthermore, both Stanisiewski et al. (1985) and Dahl et al. (1997) reported actual mean yield increases of 2.2 kg/day in response to LDPP provided by fluorescent and metal halide lamps (350 lux), respectively. Considering the prototype nature of the light mask used for this study and the frequent requirement to clear the light source of faecal debris, the increased yield observed in multiparous animals is encouraging. Additionally, the level of the increase observed in light-mask-wearing cows is also encouraging given that an increase of only 6.5% was previously reported (Dahl et al., 1997; Dahl et al., 2000). Peters et al. (1978) was the first to report that a lighting regime consisting of 16L:8D increased daily milk yield. By 10 days post-partum, the authors report that light-treated individuals produced on average 1.7 kg more than their untreated counterparts, and that by day 20 this figure had risen to 3.1 kg. This means that over the first 60 days of lactation extended photoperiod caused a 10% increase in yield (33.5±0.4 vs. 30.5±0.4 kg). With improved design and durability of the cow light mask, milk yield increases of 10% and above are potentially attainable.

Our results showed no effect of light treatment on primiparous cows and is supported by previous findings that first-calf heifers often do not respond to LDPP treatment (Marcek and Swanson, 1983; Vanbaale et al., 2005). However, this does not mean that there would not be a potential application for our mobile lighting technology in primiparous cows. Previously, weaned calves (86±2 d of age) maintained under LDPP until puberty tended (P<0.1) to produce more milk than calves maintained under natural photoperiod (Rius and Dahl, 2006). The authors report that a limitation of their study was the small number of animals per treatment group (Rius and Dahl, 2006). It is hypothesized that a future trial utilising higher animal numbers would yield results of greater significance. As milk yields of primiparous cows tend to be lower compared to subsequent lactations (Ray et al., 1992), increasing yields at first lactation may be of economic value to dairy farmers.

Additional Applications for a Bovine Light (e.g. Blue Light) Headpiece (e.g. Mask) (a Summary is Provided in Table 1)

### Puberty Advancement

Long-day photoperiod facilitates earlier breeding and thus, earlier calving (Rius and Dahl, 2006). Getting heifers to produce their first calf as early as possible is of economic interest to farmers as it means they can reap the rewards of their productive performance sooner and increase lifetime production rates. To optimise dairy farm profitability, it is therefore advisable to aim for early calving (Hultgren et al., 2011) while ensuring that the animals are sufficiently developed.

Hansen et al. (1983) reported that the onset of puberty could be hastened by exposing pre-pubertal heifers (Angus/Angus cross), born between February and July, to 18 hours of light and 6 hours of darkness (18L:6D) from 22 weeks of age until puberty. The supplementary light treatment resulted in earlier first ovulations and estrus. Age at first estrus was reduced by an average 38 days, occurring between 318-367 days old. First conception was achieved around 380 days, compared to 396 for animals exposed to natural photoperiod. The authors also noted changes in ovarian development, reporting greater ovarian growth rate and volume at 24 weeks of age in light treated individuals (Hansen et al., 1983). Tucker et al. (1984) note that provision of 16L:8D hastens the rise in testosterone and progesterone concentrations in pre-pubertal bulls and peri-pubertal heifers, which may be the reason for the earlier occurrence of first estrus and ovulation observed by Hansen et al. (1983). Similar results were reported by Kassim et al. (2008) for buffalo heifers receiving 16L:8D at 162 lux provided by tungsten lamps. They noted sharp declines in plasma MT concentrations, raised LH levels, as well as higher levels of prostaglandin (PGF2α) and estradiol-17β (E2) during estrus hours. Additionally, increased frequency of estrus behaviour resulted in improved noticeability of estrus. Light-treated heifers also had higher body weights at puberty (208±11.2 vs. 201±10.2 kg) and first ovulation (249±11.6 vs. 240±10.2 kg). As age at puberty is associated with body weight gain (Laster et al., 1972) it is unsurprising that the buffalo heifers were younger at puberty (10.5 vs. 11.7 months), and first ovulation (12.6 vs. 13.3 months), compared to their control counterparts. This implies photoperiodic influence on a heifer's sexual maturity.

Leptin is considered to be a metabolic signal involved in enhancing GnRH and LH secretion, as well as ovarian function by acting on the hypothalamic-pituitary-ovarian axis (Campbell et al., 1999). Roy et al. (2016) found that extension of photoperiod by only 4 hours between December and February (160 lux) resulted in significantly higher plasma leptin concentrations (616±99.7 pg/ml) compared to natural photoperiod (413±56.4 pg/ml). The authors hypothesized that the improved feed efficiency and average daily gain, which were also observed in the light-treated group, influenced leptin concentrations. Therefore, the impact of photoperiod on feed efficiency, average daily gain and subsequently leptin secretion may have been causal to 6 out of 7 Murrah buffalo heifers having attained puberty by the end of the experiment, compared to 4 out of 7 in the control group (Roy et al., 2016).

Furthermore, Sanchez-Barcelo et al. (1991) noted that when pre-pubertal Holstein heifers were fed MT during long-day photoperiod it reduced the growth of mammary parenchymal tissue. As light is inhibitory to MT secretion it is hypothesized that extended photoperiod positively influences mammary development and thus milk yield (Sanchez-Barcelo et al., 1991).

In summary, pre-pubertal photoperiod manipulation emerges as an effective non-invasive technique to promote lean growth and accelerate the onset of puberty without potential negative consequences on mammary development. Our results from Studies 1 and 2 indicate that photoperiod manipulation using a light mask that delivers blue light to one eye offers a management tool to enhance lean growth and accelerate puberty in the bovine.

### Fertility and Calving

Generating a faster return to reproductive competence (estrus cyclicity) after calving may also be possible using extended photoperiod, as it has been noted that prolonged return times occur in winter-calvers compared to summer-calvers (Hansen and Hauser, 1983). Exposure to extended photoperiod of 125 lux from white fluorescent lamps providing 7 watt/m² has been reported to increase conception rates, reduce service periods and reduce the number of AIs per animal (Steiger and Mehlhorn, 1976 in Reksen et al., 1999).

A survey on Norwegian Red Cattle heifers conducted by Reksen et al. (1999) found a 4 day reduction in calving interval (time between two calves being born) and days open (time between calving and conceiving again). This was achieved using >12 h photoperiod and dim illumination at night during winter, with fluorescent lamps providing 2.8 watt/m². Average light intensity was 36 lux, ranging between 4 to 160 lux at the feed alley. Furthermore, the authors observed a 3.1% increase in 60-day non-return rate, which means that more cows did not return to estrus within 60 days after breeding and were therefore considered pregnant. At calving, individuals previously exposed to extended photoperiod tended to be 6.6 days younger on average (Resken et al., 1999).

Interestingly, a study conducted by Andreu-Vázquez et al. (2012) in high-producing Spanish dairy cattle found that the incidence of twin-pregnancy, following various estrus synchronization protocols prior to artificial insemination (AI), was reduced by factors of 0.65 to 0.71 when AI was performed during the longer daylight hours of summer months. In contrast, risk was increased when conception occurred during the decreasing daylight hours of the autumn/winter season. The authors recorded twin pregnancies in 361 individuals (17.9%), and noted that risk was increased by a factor of 1.1 for each increase in lactation number. These findings are supported by Del Río et al. (2007), who noted increased number of twin-births when conception occurred during August and October.

The occurrence of twin-births has been rising steadily over the years. This is due to the dairy and beef industry's interest in shorter gestation lengths and increasing the number of calves produced per cow. However, issues associated with twin-pregnancies and twin-births include increased fetal mortality, lower conception rates, longer postpartum intervals, retained placenta and dystocia (Echternkamp and Gregory, 1999). As this requires more intensive management and veterinary attention, it can result in significant economic losses (Echternkamp and Gregory, 1999). Therefore, using extended photoperiod at the time of conception to lower the occurrence of twin-pregnancies may be an important tool for avoiding these complications, especially in autumn-calving multiparous cows. The availability of a mobile light mask may mean that reduced twinning can be attained without the need to house animals during this time.

### Growth

Extended photoperiod has been shown to increase average daily gain (ADG) and improve feed to gain ratio efficiency (Tucker et al., 1984; Roy et al., 2016). Feed efficiency increases of 9% have been observed in crossbred beef heifers that received extended winter photoperiod (16 to 19 h at 51-65 lux) administered using 250 W high-pressure sodium lamps (Kennedy et al., 2004).

A 1978 study by Peters et al. found that provision of 16L:8D (114 to 207 lux) through cool-white fluorescent lights from 06:00 until 22:00 increased not only milk yield but also body growth by 10-15%. When heart girth of pre-pubertal Holstein heifers aged between 3 to 6.5 months was measured weekly for 16 consecutive weeks between November and March, the control group showed an increase of 25 cm (114-139 cm), while the light-supplemented group achieved 29 cm (112-141 cm), a 13.8% uplift. These findings were supported by a second, similar experiment during which actual body weight was measured weekly over 22 weeks (Peters et al., 1978). This resulted in a 9.3% increase in ADG in animals exposed to LDPP compared to controls. It must be noted however, that these results were achieved between autumn and spring. A third experiment that was carried out between May and August, when natural photoperiod was >13.6 hours of light, daily weight gains did not differ significantly with supplemental light treatment (0.89 vs. 0.90 kg) (Peters et al., 1978). This indicates that best average daily gain results in response to LDPP are achieved during fall, winter and into early spring.

According to Tucker et al. (1984), photoperiodic stimulation of growth seems to be gonad-dependent in cattle. The authors base their opinion on the fact that peri-pubertal heifers experience greater live weight gains in response to increased photoperiod compared to pre-pubertal individuals. However, with advancement towards puberty the % increase in ADG increases. The authors report that intact Holstein bulls achieved a 9.8% increase in ADG under 16L:8D conditions, whereas immature bull calves or steers showed no changes in body growth. The change in secretion of reproductive hormones in the peri-pubertal period of cattle may be associated with the anabolic effects previously described and is consistent with gonad-dependency (Tucker et al., 1984).

Aside from gonadal influence, the authors also note that LDPP caused heifers with initial lower body weights to increase growth by 9.2%, while no significant effect was observed in larger animals (Tucker et al., 1984). Best growth results were achieved when light treatment was started at a body weight of 110 kg and continued through puberty.

Roy et al. (2016) also noted comparatively greater ADG in Murrah buffalo heifers exposed to 4 hours extended photoperiod (14.5L:9.5D) at 160 lux between December and February. Therefore, it seems that light treatment enables initial ‘poor doers’ to catch up on growth, but may have no significant impact on individuals that are already growing at their biological maximum rate and have reached certain weight thresholds at different developmental stages.

Interestingly, Rius and Dahl (2006) also found that extended photoperiod (16L:8D) from weaning until attainment of puberty caused heifers to be heavier (692 vs. 637 kg) and also taller at parturition (2.4 cm greater wither height, 2.8 cm greater hip height), in addition to producing more milk during their first lactation. The authors infer that breeding at a younger age can be successful without limiting milk yield or skeletal development, which would be a common concern among farmers, because extended photoperiod seems to positively affect both growth and final stature as well as capacity for increased milk production. We hypothesize that the development of a bovine light mask that provides LDPP will permit similar benefits for weight gain and growth but with the reduced management costs associated with pasture maintenance of the animals.

### Carcass Composition

Tucker et al. (1984) suggest that extended photoperiod causes more nutrients to be diverted to protein accretion, while reduced daylight hours (8L:16D) increase fat deposition by 8% or more in post-pubertal heifers. Petitclerc et al. (1984) also found that 16L:8D, combined with a high plane of nutrition aimed at achieving >1 kg/d growth, increased the 9-10-11^{th} rib section's protein content of heifers by 11% to a total of 16.2%. Gain in estimated carcass weight was 0.61 kg/day, an increase of 9.8%, compared to 8L:16D with the same plane of nutrition.

In a previous study by Petitclerc et al. (1983), 16L:8D increased body growth rate between 10-16%, irrespective of whether a low or high plane of nutrition was fed, due to improved feed conversion efficiency. According to Waldman et al. (1971), a high plane of nutrition causes a decrease in carcass protein percentage, but in the 1984 study by Petitclerc et al. extended photoperiod seems to have been capable of overcoming this issue. Kennedy et al., (2004) also reported a 15% reduction in subcutaneous back fat after 156 days of supplemental light treatment.

These results indicate that extended photoperiod may be influential in the production of leaner carcasses. Importantly, with access to a light mask as described here, these carcass improvements can now likely be attained for grass-fed beef producers.

### Milk Composition

In general, milk composition seems to be unaffected by photoperiodic stimulation (Peters et al., 1978). However, a study by Aharoni et al. (2000) found that cows maintained under natural photoperiod and calving in January, when days are short and MT levels are high, produced on average 1.9 kg/day more milk, 0.27% more fat and 0.08% more protein than cows calving in July. These findings suggest that lengthening days stimulate these compositional differences as lactation progresses. IgG levels and volume of colostrum seem unaffected by photoperiod (Morin et al., 2010).

### Semen Quality

Roussel et al. (1964) exposed bulls to 14 h of incandescent light (323±32 lux) from May to September and compared them to a matched control group. Both initial progressive motility and concentration of sperm were monitored during the 20 week period following the first of May. While declines in both parameters were observed, the decline was significantly less for bulls exposed to supplementary artificial light. Serum protein-bound iodine (PBI) levels per 100 ml, an indicator of thyroid function, was significantly higher for the light-treated group (10.12 vs. 8.20 μg). The authors inferred that photoperiod was stimulatory to thyroid activity, which they stated was involved in metabolic processes and indirectly associated with semen quality (Roussel et al., 1964).

In a previous study (Roussel et al., 1963) involving 12 dairy bulls, 409±54 lux (treatment) and 108±86 lux (control) were used and animals were divided into three groups: natural photoperiod (Group 1; control), natural+supplemental light (Group 2) and supplemental light+increased temperature and humidity (Group 3). Mean progressive semen motility was higher where supplemental light was used to extend daylength, but results did not significantly differ between groups 2 and 3, indicating that supplemental light was the primary influencer and not temperature or humidity. Photoperiodic treatment lowered the mean percentage of abnormal spermatozoa by approx. 3.9% (11.5 vs. 15.4%), caused a small increase in semen metabolic activity and improved mean spermatozoa viability (38.5% vs. 33.4%) when stored at 5° C. for 72 hours. These results may have contributed to the finding of a higher percentage of shippable ejaculate from light-treated bulls (ca. 54% vs. 18%). It was concluded that photoperiod reduces the loss of semen quality associated with heat and humidity. The authors also implied that it may affect germ cell output (Roussel et al., 1963). It must be noted that sample sizes for the above experiments were small and that animals were grouped without regard for age and breed so future studies are needed to support these initial findings. Nevertheless, a bovine light mask delivering optimum timed lighting to extend daylength may provide an attractive alternative to fixed lighting in bull housing for improved semen characteristics.

### Alternative Lighting Signals

It has been suggested that pulses of light emitted 14 to 17 hours after dawn (or 8 to 10 hours after dusk) are capable of mimicking long-day photoperiod and stimulating growth in cattle (Tucker et al., 1984). A lighting regime of 6L:8D:2L:8D also achieved the same increase in basal prolactin secretion (418% after 6 weeks) as did exposure to 16L:8D (Petitclerc et al., 1983). It is therefore feasible that a light mask programmed to emit blue light during the light-sensitive phase in the cow, 8-10 hours after dusk, could similarly mimic 16L:8D and achieve the observed milk yield increases described in Study 2. This method of simulating LDPP could potentially be used for all other LDPP applications discussed in this document.

### Dry Period Management

Individuals maintained under short-day photoperiod (SDPP) during their dry period have higher prolactin levels and increased milk yield during their subsequent lactation (Lacasse et al., 2014). Increased hours of darkness during this time is thought to prime the photoperiodic responsiveness observed during light treatment and prevent refractoriness (Dahl et al., 2000). Furthermore, Auldist et al. (2007) found that MT implants increased fat, protein and casein content, but reduced lactose concentration in milk. According to Dahl and Petitclerc (2003), primiparous individuals also show improved performance when exposed to SDPP during the latter stages of gestation followed by LDPP during lactation. They also suggest that reduced light during the dry period benefits immune function by increasing lymphocyte proliferation (Dahl and Petitclerc, 2003). Providing SDPP may be difficult for autumn calvers in the northern hemisphere as their dry period spans from August to September when the necessary hours of darkness are absent in nature and higher milk yields may have to be achieved through more expensive nutritional management. However, there may be potential to adapt the current cow light mask design such that light levels to the eyes are limited later in the day.

### CONCLUSION

These studies have demonstrated that 225 lux delivered in the form of short-wavelength (468 nm) blue light via a head-worn light mask to a single eye can effectively suppress MT to the same low levels observed under indoor housing that provides ˜250 lux white light. We provide the first evidence that wearing this technology to provide LDPP successfully increases milk yield by 8.6% in multiparous cows over the first 12 weeks of lactation. Finally, we suggest that use of this wearable light mask technology for bovines will similarly achieve the multitude of positive benefits associated with provision of LDPP via fixed lighting to animals in indoor environments. These benefits include but are not limited to the following list of applications (a summary is provided in Table 1):
Increased milk yield (8-11%) in multiparous cows
Improved milk production at first lactation in primiparous cows
Improved persistency in milk production
Faster onset of puberty, earlier first conception, improved ovarian growth
Faster rise in circulating gonadotropins
Increased frequency of estrus behavior
Higher body weights at puberty
Improved mammary development
Improved feed efficiency
Greater body growth and total weight gain
Increased heart girth
Increased average daily gain (ADG)
Increases in growth of pre-pubertal heifers with initial low body weights
Heavier heifers at parturition
Taller heifers at parturition
Increased carcass protein content
Reduction in carcass subcutaneous fat
Faster return to reproductive competence post-partum
Increased conception rates and reduced AIs per animal
Reduction in calving interval
Increases in 60-day non-return rate
Younger first calvers
Reduced risk of twin pregnancies
Slowed decline in semen viability and concentration as a result of high temperature and humidity
Improved semen quality

In summary, the applications we emphasize are the use of extended daily blue light to one eye for increasing milk yield, improved feed efficiency, growth and higher average daily weight gains and improved fertility especially in relation to early puberty onset and reduced calving interval.

TABLE 1
Summary of past literature and potential applications
of light headpiece (e.g. mask) for bovines, e.g. cows.

| | | | | | |
|---|---|---|---|---|---|
| | Animal | | Light | Light | |
| Effect | Type | Photoperiod | Intensity | System | Source |

Puberty Advancement

| | | | | | | |
|---|---|---|---|---|---|---|
| ✓ | Faster onset of | Angus/Angus | 18L:6D | Unknown | 24 | Hansen |
| | puberty | cross beef | | | fluorescent | et al. |
| ✓ | First estrus ca. 38 | heifers | | | blubs | (1983) |
| | days earlier | | | | 40 W each | |
| | (occurring between | | | | 1.6 m | |
| | 318-367 days old) | | | | above eye | |
| ✓ | First conception 16 | | | | level | |
| | days earlier | | | | Between | |
| | (occurring around | | | | 03:00 and | |
| | 380 days old, | | | | 21:00 | |
| | compared to 396) | | | | From 22 | |
| ✓ | Greater ovarian | | | | weeks of | |
| | growth rate and | | | | age to | |
| | volume at 24 weeks | | | | puberty | |
| | old | | | | | |
| ✓ | Faster rise in | Pre-pubertal | 16L:8D | Unknown | Unknown | Reviewed |
| | testosterone and | bulls | | | | in Tucker |
| | progesterone levels | Peri-pubertal | | | | et al. |
| | | heifers | | | | (1984) |
| ✓ | Declined plasma | Buffalo | 16L:8D | 162 lux | Tungsten | Kassim et |
| | melatonin and raised | heifers | | | lamps | al. (2008) |
| | LH | | | | Highest at | |
| ✓ | Higher prostaglandin | | | | 2.5 m | |
| | (PGF₂α), higher | | | | October to | |
| | Estradiol-17P (E2) | | | | March | |
| | during estrus hours | | | | | |
| ✓ | Increased frequency | | | | | |
| | of estrus behaviour | | | | | |
| | (improved | | | | | |
| | noticeability of | | | | | |
| | estrus) | | | | | |
| ✓ | Higher body weights | | | | | |
| | at puberty (208 ± | | | | | |
| | 11.2 vs. 201 ± 10.2 | | | | | |
| | kg) and first ovulation | | | | | |
| | (249 ± 11.6 vs. 240 ± | | | | | |
| | 10.2 kg) | | | | | |
| ✓ | Younger at puberty | | | | | |
| | (10.5 vs. 11.7 | | | | | |
| | months) and first | | | | | |
| | ovulation (12.6 vs. | | | | | |
| | 13.3 months) | | | | | |
| ✓ | Improved mammary | Pre-pubertal | 16I:8D | 68 lux | High | Sanchez- |
| | development | dairy | | | pressure | Barcelo |
| | (decreased | Holstein | | | sodium | et al. |
| | intraparenchymal fat, | heifers not | | | lamps | (1991) |
| | increased number or | fed | | | 1.2 m | |
| | mammary | melatonin | | | above the | |
| | parenchymal cells) | | | | floor | |
| | | | | | Between | |
| | | | | | 06:00 and | |
| | | | | | 22:00 | |
| | | | | | May to | |
| | | | | | August | |

Growth

| | | | | | | |
|---|---|---|---|---|---|---|
| ✓ | 9% feed efficiency | Spring- | 16 to 19 h | 51-65 lux | 250 W high- | (Kennedy |
| | increase | born | light | | pressure | et al., |
| | | dairy/beef | | | sodium | 2004) |
| | | crossbred | | | lamps | |
| | | heifers | | | Mounted at | |
| | | Simmental, | | | height of | |
| | | Charolais, | | | 3.2 m + | |
| | | Limousin, | | | wide-beam | |
| | | Angus, | | | floodlights | |
| | | Gelbvieh, | | | Winter | |
| | | Herefords | | | | |
| ✓ | 10-15% greater body | Holstein | 16L:8D | 114-207 | Cool-white | Peters et |
| | growth | dairy | | lux | fluorescent | al. (1978) |
| ✓ | 13.8% increase in hearth | heifers | | | lights | |
| | girth (29 vs. 25 cm) | | | | Between | |
| ✓ | 9.3% increase in | | | | 06:00 and | |
| | average daily gain | | | | 22:00 | |
| ✓ | 9.6% greater total | | | | Fall to | |
| | weight gain after 22 | | | | spring | |
| | weeks (126 vs. 114 kg) | | | | | |
| ✓ | 9.8% increase in average | Holstein | 16L:8D | Unknown | Unknown | Tucker et |
| | daily gain for intact bulls | dairy cattle | | | | al. (1984) |
| ✓ | 9.2% increase in growth | | | | | |
| | in pre-pubertal heifers | | | | | |
| | with initial low body | | | | | |
| | weights (110 kg) | | | | | |
| ✓ | Heifers 8% heavier at | Dairy | 16L:8D | 350 lux | Metal | Rius & |
| | parturition (692 vs. | heifers | | | halide | Dahl |
| | 637 kg) | | | | lamps | (2006) |
| ✓ | Heifers taller at | | | | October to | |
| | parturition (2.4 cm | | | | January | |
| | greater wither height, | | | | | |
| | 2.8 cm greater hip | | | | | |
| | height) | | | | | |

Carcass Composition

| | | | | | | |
|---|---|---|---|---|---|---|
| ✓ | 11% increase in 9-10- | Pre-pubertal | 16L:8D | Unknown | Unknown | Petitclerc |
| | 11^{th} rib section's | Holstein dairy | Combined | | | et al. |
| | protein content (to a | heifers | with a high | | | (1984) |
| | total content of | | plane of | | | |
| | 16.2%) | | nutrition | | | |
| ✓ | 9.8% increase in | | | | | |
| | estimated carcass | | | | | |
| | weight gain (0.61 | | | | | |
| | kg/day) | | | | | |
| ✓ | 15% reduction in | Spring-born | 16 to 19 h | 51-65 lux | 250 W | Kennedy |
| | subcutaneous back | dairy/beef | | | high- | et al. |
| | fat after 156 days | crossbred | | | pressure | (2004) |
| | | heifers | | | sodium | |
| | | Simmental, | | | lamps | |
| | | Charolais, | | | Mounted | |
| | | Limousin, | | | at height | |
| | | Angus, | | | of 3.2 m + | |
| | | Gelbvieh, | | | wide-beam | |
| | | Herefords | | | floodlights | |
| | | | | | Winter | |

Milk Yield

| | | | | | | |
|---|---|---|---|---|---|---|
| ✓ | At 10 days average | Holstein | 16L:8D | 114-207 | Cool-white | Peters et al. |
| | 1.7 kg/day more | dairy | | lux | fluorescent | (1978) |
| ✓ | 20 average 3.1 | heifers | | | lights | |
| | kg/day more | | | | Between | |
| ✓ | 10% increase in yield | | | | 05:00 and | |
| | over first 60 days | | | | 21:00 | |
| | (33.5 ± 0.4 vs. 30.5 ± | | | | Fall to | |
| | 0.4 kg) | | | | spring | |
| ✓ | Slowed the natural | | | | | |
| | decline in yield | | | | | |
| | which accompanies | | | | | |
| | later lactation stages | | | | | |
| ✓ | 6.7% increase | Dairy cows | 16L:8D | Unknown | Fluorescent | Peters et al. |
| | (1.4kg) per day | (various | | | lamps | (1981) |
| | during early and late | parities) | | | October to | |
| | lactation | | | | March | |
| ✓ | 7% more persistency | 38 | 18L:6D | 254 ± 26 | High | Marcek & |
| | in 4% fat-corrected | multiparous | | lux | pressure | Swanson |
| | milk(FCM) in | Holstein | | during | sodium | (1984) |
| | mature cows | dairy cows | | the day | vapor | |
| | | 36 primiparous | | 132 ± 9 | lamps | |
| | | Holstein | | lux at | 9-16 | |
| | | dairy | | night | weeks | |
| | | heifers | | | during | |
| | | | | | winter | |
| ✓ | 2.2 kg more milk/day | Dairy cows | 16L:8D | Unknown | Fluorescent | Stanisiewski |
| | | (various | | | lamps | et al. (1985) |
| | | parities) | | | | |
| ✓ | 5 to 11% increase in | Dairy cows | 16L:8D | Unknown | Unknown | Bilodeau et |
| | milk yield | | | | | al. (1989) |
| ✓ | Increased milk yield | Dairy cows | 18L:6D | 481 lux | Unknown | Phillips & |
| | (% unknown) | | | | system | Schofield |
| | | | | | Winter | (1989) |
| ✓ | Higher persistency | Dairy cows | 6 h of light h + | Unknown | Unknown | Evans & |
| | of lactation | | 2 h light | | system | Hacker |
| | | | pulse | | From 05:00 | (1989) |
| | | | | | to 11:00 | |
| | | | | | Light pulse | |
| | | | | | from 18:00 | |
| | | | | | to 20:00 | |
| ✓ | 2.2 kg/day increase | Dairy cows | 18L:6D | 350 lux | Metal | Dahl et al. |
| | (36.1 ± 0.6 vs. 33.9 ± | | | | halide | (1997) |
| | 0.6 kg/d) | | | | lamps | |
| | | | | | From 05:30 | |
| | | | | | until 23:30 | |
| | | | | | January- | |
| | | | | | April | |
| ✓ | 1.9 kg/d more fat- | 28 | 18L:6D | 350 lux | Metal | Miller et al. |
| | corrected milk | multiparous | | | halide | (1999) |
| | (FCM) | Holstein | | | lamps | |
| | | dairy cows | | | From 05:00 | |
| | | 12 primiparous | | | until 23:00 | |
| | | Holstein | | | Winter | |
| | | dairy | | | | |
| | | heifers | | | | |
| ✓ | Greater milk yield at | Dairy | 16L:8D | 350 lux | Metal | Rius & Dahl |
| | first lactation (750 kg | heifers | | | halide | (2006) |
| | of 305-d projected | | | | lamps | |
| | fat-corrected milk) | | | | October to | |
| | | | | | January | |

Milk Composition

| | | | | | | |
|---|---|---|---|---|---|---|
| ✓ | Dry period/calving | Israeli dairy | Natural | Natural | Natural | Aharoni |
| | during short-day | cattle | | | | et al. |
| | photoperiod (January) | | | | | (2000) |
| | caused 0.27% more fat | | | | | |
| | and 0.08% more | | | | | |
| | protein | | | | | |

Fertility & Calving

| | | | | | | |
|---|---|---|---|---|---|---|
| ✓ | Faster return to | Holstein | Natural | Natural | Natural | Hansen & |
| | reproductive | dairy cows | | | | Hauser |
| | competence (estrus | Hereford | | | | (1983) |
| | cyclicity) postpartum | beef cows | | | | |
| | in summer-calvers | | | | | |
| ✓ | Increased conception | Unknown | Unknown | 125 lux | White | Steiger & |
| | rates | | | | fluorescent | Mehlhorn, |
| ✓ | Reduced service | | | | lamps | (1976) in |
| | periods | | | | Providing 7 | Reksen et |
| ✓ | Less Al per animal | | | | watt/m² | al. (1999) |
| ✓ | 4 day reduction in | Norwegian | >12 h | Average | Fluorescent | Reksen et |
| | calving interval and | Red dairy | photoperiod | 36 lux | lamps | al. (1999) |
| | days open | cattle | and dim | (Range: | Average 2.8 | |
| ✓ | 3.1% increase in 60- | | illumination | 4-160 lux) | watt/m² | |
| | day non-return rate | | at night | | Winter | |
| ✓ | 6.6 days younger at | | | | | |
| | calving | | | | | |
| ✓ | Risk of twin | Spanish | Natural | Natural | Natural | Andreu- |
| | pregnancies reduced | dairy | | | | Vazquez |
| | by factors of 0.65- | cattle | | | | et al. |
| | 0.71 when conception | | | | | (2012) |
| | occurs during natural | | | | | |
| | increasing/long-day | | | | | |
| | photoperiod | | | | | |

Semen Quality

| | | | | | | |
|---|---|---|---|---|---|---|
| ✓ | Slowed decline in | Dairy/beef | 14L:10D | 323 ± 32 | 200 W | Roussel |
| | spermatozoa | bulls | | lux | incandescent | et al. |
| | concentration linked to | Jersey, | | | patio lamps | (1964) |
| | temperature and | Holstein, | | | May to | |
| | humidity (4.02 vs. 8.39 | Guernsey | | | September | |
| | percentage units) | Hereford | | | | |
| ✓ | Greater serum protein- | | | | | |
| | bound iodine (PBI) | | | | | |
| | levels per 100 ml (10.12 | | | | | |
| | vs. 8.20 μg), which is | | | | | |
| | stimulatory to thyroid | | | | | |
| | activity and indirectly | | | | | |
| | associated with semen | | | | | |
| | quality | | | | | |
| ✓ | Extended photoperiod | | | | | |
| | may affect germ cell | | | | | |
| | output | | | | | |
| ✓ | Higher mean | Dairy bulls | 14-15 h | 409 ± 54 | 200 W | Roussel |
| | progressive semen | | OR | lux | incandescent | et al. |
| | motility | | 16 h | | patio lamps | (1963) |
| ✓ | 3.9% lower mean | | | | May to | |
| | percentage of abnormal | | | | October | |
| | spermatozoa | | | | | |
| ✓ | Lower methylene blue | | | | | |
| | reduction time, which is | | | | | |
| | an indication of semen | | | | | |
| | metabolic activity | | | | | |
| ✓ | Superior mean | | | | | |
| | spermatozoa viability | | | | | |
| | (38.5% vs. 33.4%) | | | | | |
| ✓ | Higher percentage of | | | | | |
| | shippable ejaculate | | | | | |
| | (ca. 54% vs. 18%) | | | | | |

The novel eco-innovative mobile lighting technology described and tested for the first time here, represents significant progress in satisfying the Agri-Food industry's need for a new sustainable, environmentally sensitive and innovative technology for increasing individual animal productivity to meet rising demands for milk and beef. The solution—a non-invasive, therapeutic light mask product for the bovine that will provide optimized timed lighting and permit dairy and beef farmers to take advantage of light's beneficial impact on milk yields, productivity and growth without consequential environmental impacts.

### Summary of Some Aspects

It has been shown that a light mask administering blue LED light to a single eye suppresses melatonin and increases milk yield in dairy cows: applications exist for promotion of: growth, carcass composition, puberty onset and fertility.

It has been shown that blue light from light-emitting diodes (LEDs) directed at a single eye elicits a dose-dependent suppression of melatonin in dairy cows.

An objective was to determine the minimum light intensity directed at a single eye required to suppress melatonin (MT) levels to concentrations observed under indoor lighting systems in dairy cows. Following a 14-day environmental conditioning period comprising 8 hours of light, 16 hours of dark (LD8:16), where mean (±SD) white light intensity levels by day (08:00-16:00) at eye level were 237±68 Lux, five non-lactating Holstein-Friesian cows were exposed to each of the following light intensities for 8 additional hours (16:00-00:00) using a 5×5 Latin Square design; <1 Lux; 70 Lux; 125 Lux; 175 Lux and 225 Lux. Light was administered via head worn masks fitted with LEDs emitting short wavelength blue light (465 nm) to the right eye. Each treatment night was followed by a break night, where the animals again received LD8:16. Two days after completion of the different light intensity treatments, all cows were exposed to the indoor lighting system until 00:00 (LIGHTS ON). Blood samples were collected from indwelling jugular catheters at 16:00, 17:00, 18:00, 20:00, 23:00, 00:00 and 01:00 on treatment nights and at 18:00 and 22:00 on break nights. Plasma samples were assayed for MT by radioimmunoassay. MT data were log transformed and analyzed using mixed models with treatment, period, hour and treatment×hour as fixed effects and cow as a random effect. A dose-dependent effect of treatment on mean circulating MT concentrations (and 95% CI) between 16:00 and 00:00 was observed [9.1 (6.8, 12.2), 4.9 (3.7, 6.6), 4.4 (3.2, 5.8), 3.3 (2.5, 4.4), 1.8 (1.4, 2.5) and 1.9 (1.4, 2.5) ng/ml for 0, 70, 125, 175, 225 Lux and LIGHTS ON treatments, respectively]. Only the 225 Lux mask treatment did not differ from LIGHTS ON (P>0.05), and hence 225 Lux is the minimum intensity required. Further studies may examine the effect of photoperiod manipulation in grazing animals.

Study 1 investigated the minimum blue light intensity directed at a single eye required to suppress melatonin (MT) levels to concentrations observed under indoor lighting systems in dairy cows. Study 2 investigated if administration of the minimum identified blue light intensity delivered via head worn light masks to extend natural day length could influence milk yield in lactating, pasture-maintained dairy cows. Study 1: Following a 14-day environmental conditioning period comprising 8 hours of light, 16 hours of dark (8L:16D), where mean (±SD) white light intensity levels by day (08:00-16:00) at eye level were 237±68 lux, five non-lactating Holstein-Friesian cows were exposed to each of the following light intensities for 8 additional hours (16:00-00:00) using a 5×5 Latin Square design; <1 lux; 70 lux; 125 lux; 175 lux and 225 lux. Light was administered via head worn masks fitted with LEDs emitting short wavelength blue light (465 nm) to the right eye. Each treatment night was followed by a break night, where the animals again received 8L:16D. Two days after completion of the different light intensity treatments, all cows were exposed to the indoor lighting system until 00:00 (LIGHTS ON). Blood samples were collected from indwelling jugular catheters at 16:00, 17:00, 18:00, 20:00, 23:00, 00:00 and 01:00 on treatment nights and at 18:00 and 22:00 on break nights and plasma later assayed for MT by radioimmunoassay. A dose-dependent effect of treatment on mean circulating MT concentrations between 16:00 and 00:00 was observed. Only the 225 lux mask treatment did not differ from LIGHTS ON (P>0.05) and hence 225 lux is the minimum intensity required to suppress MT to indoor lighting levels during an initial exposure. Study 2: 40 spring-calving cows were blocked for parity, calving date and Economic Breeding Index for Milk Production (EBIMilk) and randomly assigned to either a CONTROL treatment or blue light to a single eye (LIGHT) treatment from day of calving through 12 weeks of lactation (February-May 2017). The LIGHT group were fitted with light masks providing 225 lux of blue light to the right eye that activated on a timer from dusk until 00:00. A significant effect of treatment on milk production was observed (P<0.0001) with the LIGHT group producing on average 1 kg/day more milk than CONTROL. Interestingly, a treatment×parity effect was also observed (P<0.0001) with a mean increase of 2.25 kg/day of milk produced by LIGHT treatment in multiparous animals versus a non-significant decrease of 0.27 kg/day in primiparous animals (P>0.05). These studies have identified the minimum blue light intensity administered to a single eye required to acutely suppress MT to levels observed under indoor lighting regimes. They provide the first evidence of an effective application of this technology for simulating a long day photoperiod (LDPP) to significantly increase daily milk yield in grass-based multiparous dairy cows over the first 12 weeks of lactation. This document has explored additional applications of this mobile lighting technology for eliciting changes in growth, carcass composition, puberty onset and fertility in the bovine.

### Note

It is to be understood that the above-referenced arrangements are only illustrative of the application for the principles of the present invention. Numerous modifications and alternative arrangements can be devised without departing from the spirit and scope of the present invention. While the present invention has been shown in the drawings and fully described above with particularity and detail in connection with what is presently deemed to be the most practical and preferred example(s) of the invention, it will be apparent to those of ordinary skill in the art that numerous modifications can be made without departing from the principles and concepts of the invention as set forth herein.

### REFERENCES

Aharoni, Y., Brosh, A. and Ezra, E. (2000) ‘Short communication: prepartum photoperiod effect on milk yield and composition in dairy cows.’, Journal of dairy science, 83(12), pp. 2779-81.
Andreu-Vázquez, C., Garcia-Ispierto, I. and López-Gatius, F. (2012) ‘Photoperiod length and the estrus synchronization protocol used before AI affect the twin pregnancy rate in dairy cattle’, Theriogenology. Elsevier Inc., 78(6), pp. 1209-1216.
Auldist, M. J., Turner, S.-A., McMahon, C. D. and Prosser, C. G. (2007) ‘Effects of melatonin on the yield and composition of milk from grazing dairy cows in New Zealand.’, The Journal of dairy research, 74(1), pp. 52-7.
Bartness, T. J. and Goldman, B. D. (1989) ‘Mammalian pineal melatonin: A clock for all seasons’, Experientia, 45(10), pp. 939-945.
Bartness, T. J., Powers, J. B., Hastings, M. H., Bittman, E. L. and Goldman, B. D. (1993) ‘The timed infusion paradigm for melatonin delivery: What has it taught us about the melatonin signal, its reception, and the photoperiodic control of seasonal responses?’, Journal of Pineal Research, 15(4), pp. 161-190.
Benloucif, S., Guico, M. J., Reid, K. J., Wolfe, L. F., L'hermite-Balériaux, M. and Zee, P. C. (2005) ‘Stability of Melatonin and Temperature as Circadian Phase Markers and Their Relation to Sleep Times in Humans’, Journal of Biological Rhythms, 20(2), pp. 178-188.
Berson, D. M., Dunn, F. A. and Takao, M. (2002) ‘Phototransduction by Retinal Ganglion Cells That Set the Circadian Clock’, Science, 295(5557), pp. 1070-1073.
Berthelot, X., Laurentie, M., Ravault, J. P., Ferney, J. and Toutain, P. L. (1990) ‘Circadian profile and production rate of melatonin in the cow’, Domestic Animal Endocrinology, 7(3), pp. 315-322.
Bilodeau, P. P., Petitclerc, D., St. Pierre, N., Pelletier, G. and St. Laurent, G. J. (1989) ‘Effects of Photoperiod and Pair-Feeding on Lactation of Cows Fed Corn or Barley Grain in Total Mixed Rations’, Journal of Dairy Science, 72(11), pp. 2999-3005.
Brainard, G. C., Hanifin, J. P., Greeson, J. M., Byrne, B., Glickman, G., Gerner, E. and Rollag, M. D. (2001) ‘Action spectrum for melatonin regulation in humans: evidence for a novel circadian photoreceptor.’, The Journal of neuroscience: the official journal of the Society for Neuroscience, 21(16), pp. 6405-12.
Campbell, B., Gutierrez, C., Armstrong, D., Webb, R. and Baird, D. (1999) ‘Leptin: in-vitro and in-vivo evidence for direct effects on the ovary in monovulatory ruminants’, Human Reproduction, 14(1), pp. 15-16.
Casey, T., Patel, O., Dykema, K., Dover, H., Furge, K. and Plaut, K. (2009) ‘Molecular signatures reveal circadian clocks may orchestrate the homeorhetic response to lactation’, PLoS ONE, 4(10).
Dahl, G. E., Auchtung, T. L. and Kendall, P. E. (2002) ‘Photoperiodic effects on endocrine and immune function in cattle’, Reproduction (Cambridge, England) Supplement, 59, pp. 191-201.
Dahl, G. E., Buchanan, B. A. and Tucker, H. A. (2000) ‘Photoperiodic Effects on Dairy Cattle: A Review’, Journal of Dairy Science, 83(4), pp. 885-893.
Dahl, G. E., Elsasser, T. H., Capuco, A. V, Erdman, R. A. and Peters, R. R. (1997) ‘Effects of a Long Daily Photoperiod on Milk Yield and Circulating Concentrations of Insulin-Like Growth Factor-I’, Journal of Dairy Science, 80 (November), pp. 2784-2789.
Dahl, G. E. and Petitclerc, D. (2003) ‘Management of photoperiod in the dairy herd for improved production and health.’, Journal of animal science, 81 Suppl 3, pp. 11-17.
Del Río, N. S., Stewart, S., Rapnicki, P., Chang, Y. M. and Fricke, P. M. (2007) ‘An Observational Analysis of Twin Births, Calf Sex Ratio, and Calf Mortality in Holstein Dairy Cattle’, Journal of Dairy Science, 90(3), pp. 1255-1264.
Evans, N. M. and Hacker, R. R. (1989) ‘Effect of Chronobiological Manipulation of Lactation in the Dairy Cow’, Journal of Dairy Science, 72(11), pp. 2921-2927.
Fariña, S. R., Garcia, S. C., Fulkerson, W. J. and Barchia, I. M. (2011) ‘Pasture-based dairy farm systems increasing milk production through stocking rate or milk yield per cow: Pasture and animal responses’, Grass and Forage Science, 66(3), pp. 316-332.
Fernandez, D. C., Chang, Y.-T., Hattar, S. and Chen, S.-K. (2016) ‘Architecture of retinal projections to the central circadian pacemaker’, Proceedings of the National Academy of Sciences, 113(21), pp. 6047-6052.
Gander, P. H., Kronauer, R. E. and Graeber, R. C. (1985) ‘Phase shifting two coupled circadian pacemakers: implications for jet lag’, American Journal of Physiology—Regulatory, Integrative and Comparative Physiology, 249(6), pp. R704-R719.
Hanifin, J. P. and Brainard, G. C. (2007) ‘Photoreception for Circadian, Neuroendocrine, and Neurobehavioral Regulation’, Journal of Physiological Anthropology, 26(2), pp. 87-94.
Hansen, P. J. (1985) ‘Seasonal modulation of puberty and the postpartum anestrus in cattle: A review’, Livestock Production Science, 12(4), pp. 309-327.
Hansen, P. J. and Hauser, E. R. (1983) ‘Genotype×environmental interactions on reproductive traits of bovine females. III. Seasonal variation in postpartum reproduction as influenced by genotype, suckling and dietary regimen.’, Journal of Animal Science, 56(6), pp. 1362-1369.
Hansen, P. J., Kamwanja, L. A. and Hauser, E. R. (1983) ‘Photoperiod influences age at puberty of heifers.’, Journal of animal science, 57(4), pp. 985-992.
Harner, J. P. and Zulovich, J. M. (2014) ‘Application of Light Emitting Diodes in Dairy Facilities’, in High Plains Dairy Conference, pp. 103-109.
Hultgren, J., Svensson, C. and Pehrsson, M. (2011) ‘Rearing conditions and lifetime milk revenues in Swedish dairy cows’, Livestock Science, 137(1-3), pp. 108-115.
Kassim, N. S. I., Afify, A. A. and Hassan, H. Z. (2008) ‘Effect of Photoperiod Length on Some Reproductive Traits and Hormonal Profiles in Buffalo Heifers’, American-Eurasian Journal of Agricultural & Environmental Sciences, 3(4), pp. 646-655.
Kennedy, A. D., Bergen, R. D., Lawson, T. J., Small, J. A. and Veira, D. M. (2004) ‘Effects of evening feeding and extended photoperiod on growth, feed efficiency, live animal carcass traits and plasma prolactin of beef heifers housed outdoors during two Manitoba winters’, Canadian Journal of Animal Science, 84(3), pp. 491-500.
Lacasse, P., Vinet, C. M., Roy, G. and Petitclerc, D. (2014) ‘Prepartum photoperiod and melatonin feeding on milk production and prolactin concentrations of dairy heifers and cows’, J. Dairy Sci. Elsevier, 97(6), p. 3589.
Laster, D. B., Glimp, H. A. and Gregory, K. E. (1972) ‘Age and weight at puberty and conception in different breeds and breed-crosses of beef heifers.’, Journal of animal science, 34(6), pp. 1031-1036.
Lawson, T. J. and Kennedy, A. D. (2001) ‘Inhibition of nighttime melatonin secretion in cattle: threshold light intensity for dairy heifers’, Canadian Journal of Animal Science, 81(1993), pp. 153-156.
Loat, C. E. and Rhodes, E. C. (1989) ‘Jet-lag and human performance.’, Sports medicine (Auckland, N.Z.), 8(4), pp. 226-38.
Marcek, J. M. and Swanson, L. V. (1983) The Effect of Photoperiod on Milk Production and Prolactin in Holstein Dairy Cows.
Miller, A. R. E., Stanisiewski, E. P., Erdman, R. A., Douglass, L. W. and Dahl, G. E. (1999) ‘Effects of long daily photoperiod and bovine somatotropin (Trobest®) on milk yield in cows’, Journal of Dairy Science, 82(8), pp. 1716-1722.
Miyake, S., Sumi, Y., Yan, L., Takekida, S., Fukuyama, T., Ishida, Y., Yamaguchi, S., Yagita, K. and Okamura, H. (2000) ‘Phase-dependent responses of Pert and Per2 genes to a light-stimulus in the suprachiasmatic nucleus of the rat’, Neuroscience Letters, 294(1), pp. 41-44.
Moore, R. Y. and Eichler, V. B. (1972) ‘Loss of a circadian adrenal corticosterone rhythm following suprachiasmatic lesions in the rat’, Brain Research, 42(1), pp. 201-206.
Morgan, P. J. and Mercer, J. G. (1994) ‘Control of seasonality by melatonin’, Proceedings of the Nutrition Society, 53(3), pp. 483-493.
Morin, D. E., Nelson, S. V, Reid, E. D., Nagy, D. W., Dahl, G. E. and Constable, P. D. (2010) ‘Effect of colostral volume, interval between calving and first milking, and photoperiod on colostral IgG concentrations in dairy cows.’, Journal of the American Veterinary Medical Association, 237(4), pp. 420-428.
Muthuramalingam, P., Kennedy, A. D. and Berry, R. J. (2006) ‘Plasma melatonin and insulin-like growth factor-1 responses to dim light at night in dairy heifers’, Journal of Pineal Research, 40(3), pp. 225-229.
Perreau-Lenz, S., Kalsbeek, A., Pevet, P. and Buijs, R. M. (2004) ‘Glutamatergic clock output stimulates melatonin synthesis at night’, European Journal of Neuroscience, 19(2), pp. 318-324.
Peters, R. R., Chapin, L., Leining, K. B. and Tucker, H. A. (1978) ‘Supplemental Lighting Stimulates Growth and Lactation in Cattle’, Science, New Series, 199(4331), pp. 911-912.
Peters, R. R., Chapin, L. T., Emery, R. S. and Tucker, H. A. (1981) ‘Milk Yield, Feed Intake, Prolactin, Growth Hormone, and Glucocorticoid Response of Cows to Supplemented Light’, Journal of Dairy Science, pp. 1671-1678.
Petitclerc, D., Chapin, L. T., Emery, R. S. and Tucker, H. A. (1983) ‘Body growth, growth hormone, prolactin and puberty response to photoperiod and plane of nutrition in Holstein heifers.’, Journal of animal science, 57(4), pp. 892-898.
Petitclerc, D., Chapin, L. T. and Tucker, H. A. (1984) ‘Carcass Composition and Mammary Development Responses to Photoperiod and Plane of Nutrition in Holstein Heifers’, Journal of animal science, 58(4), pp. 913-919.
Phillips, C. J. C. and Schofield, S. A. (1989) ‘The effect of supplementary light on the production and behaviour of dairy cows’, Animal Production, 48(2), pp. 293-303.
Porter, P. A. and Luhman, C. M. (2002) ‘Changing photoperiod improves persistency in high producing Holstein cows’, Journal of Dairy Science, 85(Suppl. 1), p. MidWest Sect. Abstr.
Provencio, I., Rodriguez, I. R., Jiang, G., Hayes, W. P., Moreira, E. F. and Rollag, M. D. (2000) ‘A novel human opsin in the inner retina’, J Neurosci, 20(2), pp. 600-605.
Ray, D. E., Halbach, T. J. and Armstrong, D. V. (1992) ‘Season and Lactation Number Effects on Milk Production and Reproduction of Dairy Cattle in Arizona’, Journal of Dairy Science, 75(11), pp. 2976-2983.
Reddy, A. B., Field, M. D., Maywood, E. S. and Hastings, M. H. (2002) ‘Differential Resynchronisation of Circadian Clock Gene Expression within the Suprachiasmatic Nuclei of Mice Subjected to Experimental Jet Lag’, The Journal of Neuroscience, 22(7), pp. 7326-7330.
Reksen, O., Tverdal, A., Landsverk, K., Kommisrud, E., Bøe, K. E. and Ropstad, E. (1999) ‘Effects of Photointensity and Photoperiod on Milk Yield and Reproductive Performance of Norwegian Red Cattle’, Journal of Dairy Science, 82(4), pp. 810-816.
Rius, A. G. and Dahl, G. E. (2006) ‘Exposure to Long-Day Photoperiod Prepubertally May Increase Milk Yield in First-Lactation Cows’, Journal of Dairy Science, 89(6), pp. 2080-2083.
Roussel, J. D., Patrick, T. E., Kellgren, H. C. and Breidenstein, C. P. (1963) ‘Effect of Artificial Light, Temperature, and Humidity on Physiological Response of Dairy Bulls’, Journal of Dairy Science, 46(10), pp. 1125-1131.
Roussel, J. D., Patrick, T. E., Kellgren, H. C. and Guidry, A. J. (1964) ‘Influence of Incandescent Light on Reproductive and Physiological Responses of Bovine Bulls’, Journal of Dairy Science, 47(2), pp. 175-178.
Roy, A. K., Singh, M., Kumar, P. and Kumar, B. S. B. (2016) ‘Effect of extended photoperiod during winter on growth and onset of puberty in Murrah buffalo heifers’, Veterinary World, 9(2), pp. 216-221.
Sanchez-Barcelo, E. J., Mediavilla, M. D., Zinn, S. A., Buchanan, B. A., Chapin, L. T. and Tucker, H. A. (1991) ‘Melatonin suppression of mammary growth in heifers.’, Biology of reproduction, 44(5), pp. 875-879.
Shearman, L. P., Zylka, M. J., Weaver, D. R., Kolakowski, L. F. and Reppert, S. M. (1997) ‘Two period homologs: Circadian expression and photic regulation in the suprachiasmatic nuclei’, Neuron, 19(6), pp. 1261-1269.
Stanisiewski, E. P., Chapin, L. T., Ames, N. K., Zinn, S. A. and Tucker, H. A. (1988) ‘Melatonin and prolactin concentrations in blood of cattle exposed to 8, 16 or 24 hours of daily light.’, Journal of animal science, 66(3), pp. 727-734.
Stanisiewski, E. P., Mellenberger, R. W., Anderson, C. R. and Tucker, H. A. (1985) ‘Effect of Photoperiod on Milk Yield and Milk Fat in Commercial Dairy Herds’, Journal of Dairy Science, 68(5), pp. 1134-1140.
Stephan, F. K. and Zucker, I. (1972) ‘Circadian Rhythms in Drinking Behavior and Locomotor Activity of Rats Are Eliminated by Hypothalamic Lesions.’, Proc Natl Acad Sci USA, 69(6), pp. 1583-1586.
Thapan, K., Arendt, J. and Skene, D. J. (2001) ‘An action spectrum for melatonin suppression: Evidence for a novel non-rod, non-cone photoreceptor system in humans’, Journal of Physiology, 535(1), pp. 261-267.
Tucker, H. A., Petitclerc, D. and Zinn, S. A. (1984) ‘The Influence of Photoperiod on Body Weight Gain, Body Composition, Nutrient Intake and Hormone Secretion’, Journal of animal science, 59(6), pp. 1610-1620.
Vanbaale, M. J., Armstrong, D. V., Etchebarne, M. A., Mattingly, R. M. and Fiscalini, J. B. (2005) Case Study: Supplemental Light for Lactating Dairy Cows.
Waldman, R. C., Tyler, W. J. and Brungardt, V. H. (1971) ‘Changes in Carcass Composition of Holstein Steers associated with Ration Energy Levels and Growth’, Journal of animal science, 32(4), pp. 611-619.
Yamazaki, S., Numano, R., Abe, M., Hida, A., Takahashi, R., Ueda, M., Block, G., Sakaki, Y., Menaker, M. and Tei, H. (2000) ‘Resetting Central and Peripheral Circadian Oscillators in Transgenic Rats’, Science, 288(5466), pp. 682-685.
Yan, L., Takekida, S., Shigeyoshi, Y. and Okamura, H. (1999) ‘Pert and Per2 gene expression in the rat suprachiasmatic nucleus: Circadian profile and the compartment-specific response to light’, Neuroscience, 94(1), pp. 141-150.

1. A light-based method of causing a substantial suppression of melatonin production, the method comprising the step of shining blue light from an artificial blue light source into just one eye of a bovine, causing a substantial suppression of melatonin production in the bovine, sufficient to induce physiological change.

2. The method of claim 1, in which the physiological change is a non-therapeutic physiological change.

3. The method of claim 1, the method comprising directing blue light into the bovine's eye for at least one selected period of time.

4. The method of claim 1, the method including the step of providing to the bovine a combined total of natural and blue light from the artificial blue light source for at least approximately 16 hours during each 24 hour period.

5. The method of claim 1, the method including the step of keeping the bovine outdoors whilst the blue light from the artificial blue light source is shone into the just one eye of the bovine.

6. (canceled)

7. The method of claim 1, in which the blue light has a peak wavelength of from 440 to 490 nm, or in which the blue light has a peak wavelength of from 459 to 484 nm, or in which the blue light has a peak wavelength of 465 nm.

8-9. (canceled)

10. The method of claim 1, in which the artificial blue light source includes a white light source.

11. (canceled)

12. The method of claim 1, in which the artificial blue light source includes a white light LED and a blue filter.

13. The method of claim 1, in which the artificial blue light source comprises at least one blue LED.

14. The method of claim 1, in which the artificial blue light source includes a white light LED and no blue filter.

15. The method of claim 1, in which light incident on the just one eye of the bovine is blue.

16. (canceled)

17. The method of claim 1, in which the artificial light source provides a blue light intensity of from 10 to 400 lux at the just one eye of the bovine, or in which the artificial light source provides a blue light intensity of from 100 to 300 lux at the just one eye of the bovine, or in which the artificial light source provides a blue light intensity of from 200 to 250 lux at the just one eye of the bovine.

18-21. (canceled)

22. The method of claim 1, in which the blue light is directed into only a single one of the bovine's eyes at any given time.

23. (canceled)

24. The method of claim 1, in which the bovine is a cow, a bull, a heifer, a steer, a calf, an ox, or a buffalo.

25. The method of claim 1, wherein the bovine is a cow, and the physiological change includes increased milk yield from the cow.

26. The of claim 25, wherein the cow is a multiparous cow.

27. The method of claim 26, wherein the increased milk yield is from 1% to 9%, or from 1% to 11%.

28. (canceled)

29. The method of claim 1, (i) wherein the bovine is a cow, and the physiological change includes one or more of faster onset of puberty, earlier first conception, or improved ovarian growth; or (ii) wherein the physiological change includes an increased rise in circulating gonadotropins; or (iii) wherein the bovine is a cow, and the physiological change includes increased frequency of estrus behavior, or improved mammary development; or (iv) wherein the physiological change includes increased body weight at puberty, or improved feed efficiency, or greater body growth and total weight gain, or increased heart girth, or increased average daily gain (ADG); or (v) wherein the bovine is a heifer, and the physiological change includes increased growth of a pre-pubertal heifer with an initial low body weight, or a heavier heifer at parturition, or a taller heifer at parturition; or (vi) wherein the physiological change includes increased carcass protein content, or reduction in carcass subcutaneous fat; or (vii) wherein the bovine is a cow, and the physiological change includes faster return to reproductive competence post-partum, or increased conception rates and reduced artificial inseminations per animal, or reduction in calving interval, or increases in 60-day non-return rate, or younger first calvers, or reduced risk of twin pregnancies; or (viii) wherein the bovine is a bull, and the physiological change includes slowed decline in semen viability and concentration as a result of high temperature and humidity, or improved semen quality.

30-36. (canceled)

37. A device for causing a substantial suppression of melatonin production, the device comprising a headpiece fittable to a bovine's head, and including an artificial blue light source, the headpiece operable to shine blue light from the artificial blue light source into an eye of the bovine, causing a substantial suppression of melatonin production in the bovine, sufficient to induce physiological change.

38-70. (canceled)

71. A system including a device for causing a substantial suppression of melatonin production, the device comprising a headpiece fittable to a bovine's head, and including an artificial blue light source, the headpiece operable to shine blue light from the artificial blue light source into an eye of the bovine, causing a substantial suppression of melatonin production sufficient to induce physiological change, in which the light source is operable to provide blue light at the eye of the bovine, the system including a control in connection with the device, in which the control is configured to turn on the device, wherein the device is configured to direct blue light into the bovine's eye for at least one selected period of time.

72-77. (canceled)
